(19) United States
(12) Patent Application Publication
(10) Pub. No.: US 2025/0029681 A1
(43) Pub. Date: Jan. 23, 2025

(54) SYSTEMS AND METHODS FOR CELL-TYPE IDENTIFICATION

(71) Applicant: St. Jude Children's Research Hospital, Inc., Memphis, TN (US)

(72) Inventors: Paul GEELEHER, Memphis, TN (US); Asif ZUBAIR, Memphis, TN (US)

(73) Assignee: St. Jude Children's Research Hospital, Inc., Memphis, TN (US)

(21) Appl. No.: 18/707,385

(22) PCT Filed: Nov. 3, 2022
(86) PCT No.: PCT/US2022/048781

§ 371 Date: May 3, 2024

Related U.S. Application Data
(60) Provisional application No. 63/278,297, filed on Nov. 11, 2021.
(60) Provisional application No. 63/275,577, filed on Nov. 4, 2021.

(51) Int. Cl. G16B 45/00; G06T 7/00; G16B 25/10

(52) U.S. Cl. CPC G16B 45/00; G16B 25/10; G06T 7/0014

(57) ABSTRACT

An exemplary embodiment of the present disclosure provides systems and methods for mapping a location of cell types within a tissue sample. The system may include one or more processors, a non-transient memory in communication with the one or more processors storing instructions that when executed by the one or more processors are configured to perform method steps. The method may include receiving a tissue sample, capturing image data of the tissue sample, extracting a plurality of nucleic molecules from the tissue sample, and generating spatially resolved transcriptomic data from the extracted plurality of cellular analytes. The method may include determining cell-type reference data and generating a feature map of the tissue sample that includes a final inferred cell type compositional map for each tissue region of the tissue sample based on the spatially resolved transcriptomics data, the cell-type reference data, and the mapping estimate of cell types.

### CROSS-REFERENCE TO RELATED APPLICATIONS

This application claims priority to and the benefit under Article 8 of the Patent Cooperation Treaty of U.S. Provisional Patent Application No. 63/275,577 filed 4 Nov. 2021 and U.S. Provisional Patent Application No. 63/278,297, filed 11 Nov. 2021, the entirety of each of which are incorporated herein by reference as if set forth herein in their entirety.

### FEDERALLY SPONSORED RESEARCH STATEMENT

This invention was made with government support under grant number GM138293 awarded by the National Institutes of Health. The government has certain rights in the invention.

### FIELD OF THE DISCLOSURE

The various embodiments of the present disclosure relate generally to generating spatially resolved transcriptomics data for a tissue sample, and more particularly to enhancing spatial transcriptomics data with mapping estimates derived from image data to generate feature maps of tissue samples that identify cell type by tissue region of a tissue sample.

### BACKGROUND

Spatial transcriptomics is a burgeoning field of study that describes a range of systems and methods designed to assign cell types (identified by mRNA sequence data) to their location in histological tissue samples, as well as measuring gene activity in a tissue sample and measuring where within the tissue sample the gene activity is occurring. Spatial transcriptomics is a recent advancement of the transcriptomics field. Previous transcriptomics methods could identify cell subpopulations within a tissue sample but were not capable of capturing cell spatial distributions nor could previous methods reveal cellular interactions between cell subpopulations within a given tissue sample.

Additionally, long-established pathology images, such as hematoxylin and eosin (“H&E”) stains, are typically collected on tissue samples as part of established spatial transcriptomics protocols. Recent advances in artificial intelligence and deep learning models have enabled systems that can computationally annotate and interpret these types of biological images. However, no current methodology has been described that allows spatial transcriptomics data and AI annotated images to be leveraged jointly.

Accordingly, there is a need for a novel methodology that can jointly leverage AI annotated pathology images with spatial transcriptomics data to improve inferences of cell-type composition over either class of data alone. The disclosed embodiments are directed to these and other considerations.

### BRIEF SUMMARY

Certain disclosed embodiments provide systems and methods for mapping a location of cell types within a tissue sample. The disclosed embodiments provide for a conceptually novel methodology termed Guiding-Image Spatial Transcriptomics (“GIST”), that can jointly leverage spatial transcriptomics data and AI annotated tissue images. The method may include receiving the tissue sample. The tissue sample may include a plurality of cell types distributed over a plurality of tissue regions of the tissue sample. The method may include capturing image data of the tissue sample, and generating, using the captured image data, a mapping estimate of cell types for each tissue region of the tissue sample. The method may include extracting a plurality of nucleic acid molecules from the tissue sample. Each of the plurality of nucleic acid molecules extracted from the tissue sample may be associated with a respective cell type of the plurality of cell types present within the tissue sample. The method may include generating spatially resolved transcriptomic data from the extracted plurality of nucleic acid molecules for each tissue region of the tissue sample. The spatially resolved transcriptomic data may be generated using a spatial transcriptomics platform to process the extracted plurality of nucleic acid molecules. The method may include determining cell-type reference data. The cell-type reference data may include gene expression by cell type for each cell type of the tissue sample. The method may include generating an output of a feature map of the tissue sample. The feature may be a final inferred cell type compositional map for each tissue region of the tissue sample. The final inferred cell type compositional map may be based on the spatially resolved transcriptomics data, the determined cell-type reference data, and the mapping estimate of cell types.

In another aspect, a system for mapping a location of cell types within a tissue sample is disclosed. The system may include one or more processors, and a non-transient memory in communication with the one or more processors storing instructions, that when executed by the one or more processors are configured to cause the system to perform steps of a method. The method may include capturing image data of the tissue sample, and generating, using the captured image data, a mapping estimate of cell types for reach tissue region of the tissue sample. The method may include extracting a plurality of nucleic acid molecules from the tissue sample. Each of the plurality of nucleic acid molecules extracted from the tissue sample may be associated with a respective cell type of the plurality of cell types present within the tissue sample. The method may include generating spatially resolved transcriptomic data from the extracted plurality of nucleic acid molecules for each tissue region of the tissue sample. The spatially resolved transcriptomic data may be generated using a spatial transcriptomics platform to process the extracted plurality of nucleic acid molecules. The method may include determining cell-type reference data. The cell-type reference data may include gene expression by cell type for each cell type of the tissue sample. The method may include generating an output of a feature map of the tissue sample. The feature may be a final inferred cell type compositional map for each tissue region of the tissue sample. The final inferred cell type compositional map may be based on the spatially resolved transcriptomics data, the determined cell-type reference data, and the mapping estimate of cell types.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 is a block diagram of an example system 100 that may be used to generate spatially resolved cell type compositional maps using GIST, in accordance with an exemplary embodiment of the present invention.

FIG. 2 is a block diagram of an example tissue imaging platform 120, as shown in FIG. 1, with additional details.

FIG. 3 is a block diagram of an example spatial transcriptomics platform 130, as shown in FIG. 1, with additional details.

FIGS. 4A and 4B provide an overview of an example GIST methodology, in accordance with an exemplary embodiment of the present invention.

FIG. 5A provides a schematic representation of an example cell-type decomposition problem, posed as a matrix decomposition, in accordance with an exemplary embodiment of the present invention.

FIGS. 5B and 5C are boxplots showing cell type decomposition results for five methods on simulated mixture gene expression data, in accordance with an exemplary embodiment of the present invention.

FIGS. 6A through 6I provide an example of how incorporating image-derived prior information from matched immunofluorescence stains can improve cell-type decomposition estimates in spatial transcriptomics data derived from mouse brain, in accordance with an exemplary embodiment of the present invention.

FIGS. 7A through 7I provide an example of how incorporating prior information derived from deep learning models applied to matched H&E stained images can improve estimates of immune cell infiltration and abundance in spatial transcriptomics applied to breast tumors, in accordance with an exemplary embodiment of the present invention.

FIGS. 8A through 8I provide an example of how GIST model-derived cell-type annotations can provide better-than-pathologist performance, in accordance with an exemplary embodiment of the present invention.

FIG. 9 is a flowchart of an exemplary method of generating a final inferred cell type compositional map using the GIST model, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

Where values are described as ranges, it will be understood that such value includes the values of all possible sub-ranges within such ranges, as well as specific numerical values that fall within such ranges irrespective of whether a specific numerical value or specific sub-range is expressly stated. In addition, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g., “about 90%” may refer to the range of values from 81% to 99%.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, tagmentation, or other approaches. Adaptors may also be used to refer to a nucleic acid sequence or segment, such as a functional sequence. These adaptors may comprise nucleic acid sequences that may add a function, e.g., spacer sequence, primer sequencing site, barcode sequence, unique molecular identifier sequence, etc. As used herein, “Y-adapter” and “forked adapter” may be used synonymously.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina R, Pacific Biosciences (PacBio®), Oxford Nanopore R, or Life Technologies (Ion Torrent R). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

As used herein, the term, “single-cell RNA-seq” refers to high-throughput single-cell RNA-sequencing protocols. Single-cell RNA-seq includes, but is not limited to, Drop-seq, Seq-Well, InDrop ICell Bio. Single-cell RNA-seq methods also include, but are not limited to, smart-seq2, TruSeq, CEL-Seq, STRT, Quartz-Seq, or any other similar method known in the art. Multiple technologies have been described that massively parallelize the generation of RNA-seq libraries that can be used in the present disclosure.

As used herein, the term “spatial transcriptomics slide” refers to slides prepared with nucleic acid capturing probes on the surface of the slides as well as spatially barcoded oligonucleotides that provide spatial resolution to captured nucleic acids. Spatial transcriptomics slides may include technologies such as Visium, Visium HD, and Slide-Seq, although spatial transcriptomics slides are not expressly limited to these technology platforms.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle,” as used herein, generally refers to a discrete biological system derived from a biological sample. The biological particle may be a macromolecule, a small molecule, a virus, a cell or derivative of a cell, an organelle, or a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell and may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “analyte,” as used herein, generally refers to a substance or one or more chemical constituents thereof that are capable of being identified and/or measured, such as by detection (e.g., detection via sequencing). Generally, this application refers to analytes from and/or produced by cells, for example as found in tissue samples. Examples of analytes include, without limitation, DNA, RNA, synthetic oligonucleotides, the labelling agents described herein, antibodies, proteins, peptides, saccharides, polysaccharides, lipids, nucleic acids, and other biomolecules. An analyte may be a cell or one or more constituents of a cell.

Analytes may be of different types. In some examples, in a plurality of analytes, a given analyte is of a different structural or functional class from other analytes of the plurality. Examples of different types of analytes include DNA and RNA; a nucleic acid molecule and a labelling agent; a transcript and genomic nucleic acid; a plurality of nucleic acid molecules, where each nucleic acid molecule has a different function, such as a different cellular function. A sample may have a plurality of analytes of different types, such as a mixture of DNA and RNA molecules, or a mixture of nucleic acid molecules and labelling agents.

The term “spatial” refers to a location within or on a space. In some examples, the space may be a two-dimensional space. “Spatially-resolved” or “spatial-resolution” are generally used to describe the ability of a spatial analysis system to attribute, correlate or match expression of an analyte to one or more cells. High resolution is desirable and refers to the situation where expression of analytes can be ascribed to single cells.

Spatial analysis methodologies and compositions described herein can provide a vast amount of analyte and/or expression data for a variety of analytes within a biological sample at high spatial resolution. Spatial analysis methods and compositions can include, e.g., the use of a capture probe including a spatial barcode (e.g., a nucleic acid sequence that provides information as to the location or position of an analyte within a cell or a tissue sample, including a mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample, it serves as a proxy for the analyte.

Generally, the invention relates to imaging samples overlaying with biomarker information based on gene expression, also called transcriptomic data. Additionally, machine learning and deep learning techniques are utilized to assess the imaging samples to improve the identification of different cell types in the transcriptomic data. The invention provides methods that can be utilized to assess pathology-based clinical diagnostics (e.g., computer methods performed on transcriptomic data of a subject), and then treat certain tissues (e.g., therapeutic methods performed on a subject). The invention includes methods, systems, apparatuses, computer program products, among others, to carry out the following.

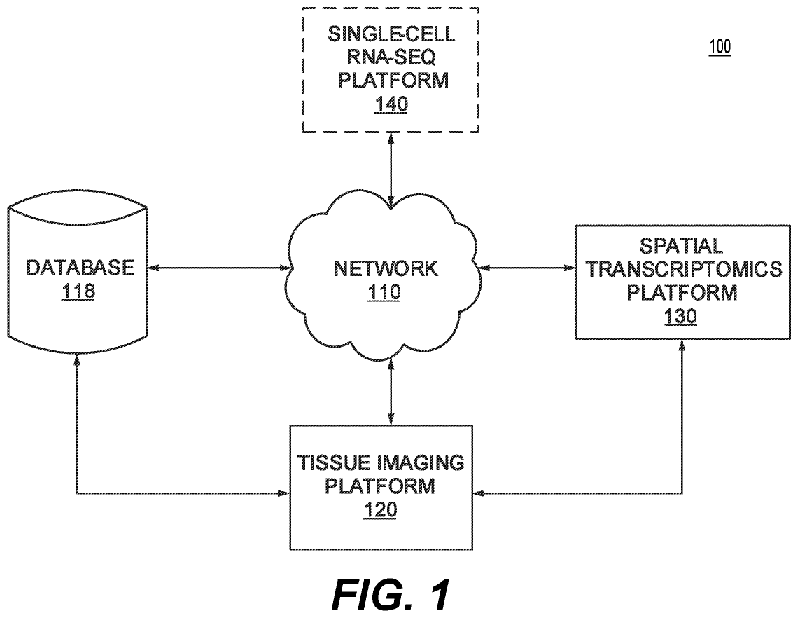

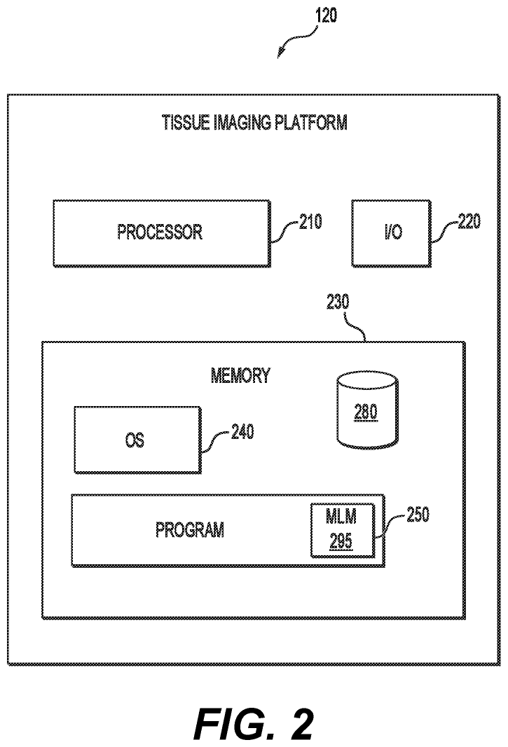

In accordance with certain disclosed embodiments, and as shown in FIG. 1, system environment 100 may include a tissue imaging platform 120 in communication with a spatial transcriptomics platform 130, a single-cell RNA-Seq platform 140, and a database 118 over network 110. Tissue imaging platform 120 may be a computing device, such as a mobile computing device (e.g., a smart phone, tablet computer, smart wearable device, portable laptop computer, voice command device, wearable augmented reality device, or other mobile computing device or fixed computing device (e.g., a desktop computer or server). An example architecture of tissue imaging platform 120 that may be used to implement one or more aspects of system 100 is described below with reference to FIG. 2.

Tissue imaging platform 120 may receive imaging data, which may be pathology images of tissue samples, that tissue imaging platform 120 may use as an input into a deep learning model to identify cell types within the tissue sample. The deep learning model implemented by tissue imaging platform 120 may be trained using annotated medical images of the same tissue sample or a similar tissue sample that includes cells of the same cell type as the instant tissue sample. The tissue imaging platform may implement one or more deep learning models, as will be described in more detail with respect to FIG. 2.

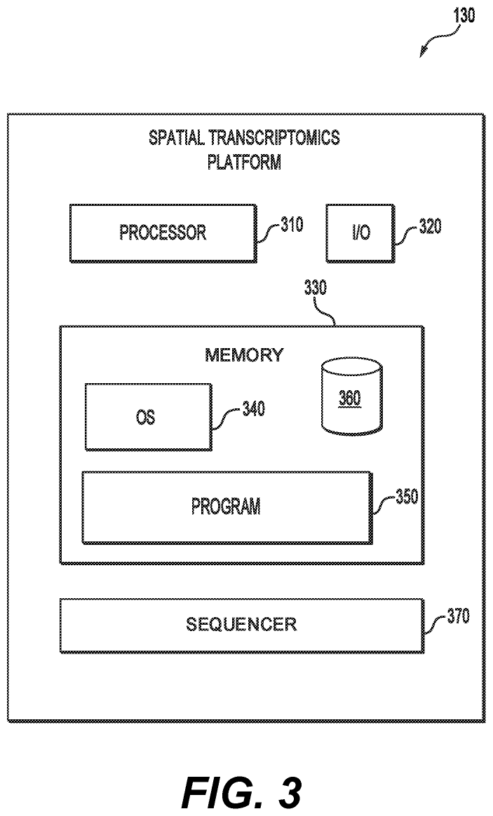

Spatial transcriptomics platform 130 may be configured to receive the tissue sample (e.g., the same tissue sample used by tissue imaging platform 120) and perform a sequence of steps to permeabilize the tissue, mark nucleic acid molecules with primers that preserve positional information of the tissue sample, and sequence the primered nucleic acid molecules to generate a spatially resolved transcriptomics data. Spatial transcriptomics platform 130 may be a computing device, such as a mobile computing device (e.g., a smart phone, tablet computer, smart wearable device, portable laptop computer, voice command device, wearable augmented reality device, or other mobile computing device or fixed computing device (e.g., a desktop computer or server). An example architecture of spatial transcriptomics platform 130 that may be used to implement one or more aspects of system 100 is described below with reference to FIG. 3.

Single-cell RNA-Seq Platform 140 may be configured to receive the tissue sample (e.g., the same tissue sample used by the tissue imaging platform 120), or a tissue sample from an adjacent tissue section, or a tissue sample from a different patient but having a similar composition of cells, and generate a single-cell RNA-seq dataset from the tissue sample.

According to some embodiments, the database 118 may be a database that stores training images (e.g., pathologist annotated images) of tissue samples used to train the deep learning model implemented by tissue imaging platform 120. The database 118 may also serve as a back-up storage device and may contain data and information that is also stored on, for example, database 280, as will be discussed with reference to FIG. 2. The database 118 may be accessed by the tissue imaging platform 120 and may be used to annotated training images used to enable certain functionality of system 100. Database 118 may continuously or intermittently receive update training images, which may be intermittently or continuously utilized by tissue imaging platform 120 to refine and/or update the deep learning model implemented by tissue imaging platform 120.

Network 110 may be of any suitable type, including individual connections via the internet such as cellular or Wi-Fi networks. In some embodiments, network 110 may connect terminals using direct connections such as radio-frequency identification (RFID), near-field communication (NFC), Bluetooth™, low-energy Bluetooth™ (BLE), Wi-Fi™, ZigBee™, ambient backscatter communications (ABC) protocols, USB, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security. One of ordinary skill will recognize that various changes and modifications may be made to system environment 100 while remaining within the scope of the present disclosure. Moreover, while the various components have been discussed as distinct elements, this is merely an example, and, in some cases, various elements may be combined into one or more physical or logical systems. According to some embodiments, database 118, tissue imaging platform 120, and spatial transcriptomics platform 130 may be directly connected in lieu of, or in addition to being in communication via network 110.

FIG. 2 is a block diagram (with additional details) of the tissue imaging platform 120, as also depicted in FIG. 1. According to some embodiments, spatial transcriptomics platform 130 and database 118 may have a similar structure and components that are similar to those described with respect to tissue imaging platform 120 shown in FIG. 2. As shown, the tissue imaging platform 120 may include a processor 210, an input/output (“I/O”) device 220, a memory 230 containing an operating system (“OS”) 240 and a program 250. In certain example implementations, the tissue imaging platform 120 may be a single server or may be configured as a distributed computer system including multiple servers or computers that interoperate to perform one or more of the processes and functionalities associated with the disclosed embodiments. In some embodiments, the tissue imaging platform 120 may further include a peripheral interface, a transceiver, a mobile network interface in communication with the processor 210, a bus configured to facilitate communication between the various components of the tissue imaging platform 120, and a power source configured to power one or more components of the tissue imaging platform 120.

A peripheral interface, for example, may include the hardware, firmware and/or software that enable(s) communication with various peripheral devices, such as media drives (e.g., magnetic disk, solid state, or optical disk drives), other processing devices, or any other input source used in connection with the disclosed technology. In some embodiments, a peripheral interface may include a serial port, a parallel port, a general-purpose input and output (GPIO) port, a game port, a universal serial bus (USB), a micro-USB port, a high definition multimedia (HDMI) port, a video port, an audio port, a Bluetooth™ port, a near-field communication (NFC) port, another like communication interface, or any combination thereof.

In some embodiments, a transceiver may be configured to communicate with compatible devices and ID tags when they are within a predetermined range. A transceiver may be compatible with one or more of: radio-frequency identification (RFID), near-field communication (NFC), Bluetooth™, low-energy Bluetooth™ (BLE), WiFi™, ZigBee™, ambient backscatter communications (ABC) protocols or similar technologies.

A mobile network interface may provide access to a cellular network, the Internet, or another wide-area or local area network. In some embodiments, a mobile network interface may include hardware, firmware, and/or software that allow(s) the processor(s) 210 to communicate with other devices via wired or wireless networks, whether local or wide area, private or public, as known in the art. A power source may be configured to provide an appropriate alternating current (AC) or direct current (DC) to power components.

The processor 210 may include one or more of a microprocessor, microcontroller, digital signal processor, co-processor or the like or combinations thereof capable of executing stored instructions and operating upon stored data. The memory 230 may include, in some implementations, one or more suitable types of memory (e.g. such as volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like), for storing files including an operating system, application programs (including, for example, a web browser application, a widget or gadget engine, and or other applications, as necessary), executable instructions and data. In one embodiment, the processing techniques described herein may be implemented as a combination of executable instructions and data stored within the memory 230.

The processor 210 may be one or more known processing devices, such as, but not limited to, a microprocessor from the Pentium™ family manufactured by Intel™ or the Turion™ family manufactured by AMD™. The processor 210 may constitute a single core or multiple core processor that executes parallel processes simultaneously. For example, the processor 210 may be a single core processor that is configured with virtual processing technologies. In certain embodiments, the processor 210 may use logical processors to simultaneously execute and control multiple processes. The processor 210 may implement virtual machine technologies, or other similar known technologies to provide the ability to execute, control, run, manipulate, store, etc. multiple software processes, applications, programs, etc. One of ordinary skill in the art would understand that other types of processor arrangements could be implemented that provide for the capabilities disclosed herein.

In accordance with certain example implementations of the disclosed technology, the tissue imaging platform 120 may include one or more storage devices configured to store information used by the processor 210 (or other components) to perform certain functions related to the disclosed embodiments. In one example, the tissue imaging platform 120 may include the memory 230 that includes instructions to enable the processor 210 to execute one or more applications, such as server applications, network communication processes, and any other type of application or software known to be available on computer systems. Alternatively, the instructions, application programs, etc. may be stored in an external storage or available from a memory over a network. The one or more storage devices may be a volatile or non-volatile, magnetic, semiconductor, tape, optical, removable, non-removable, or other type of storage device or tangible computer-readable medium.

In one embodiment, the tissue imaging platform 120 may include a memory 230 that includes instructions that, when executed by the processor 210, perform one or more processes consistent with the functionalities disclosed herein. Methods, systems, and articles of manufacture consistent with disclosed embodiments are not limited to separate programs or computers configured to perform dedicated tasks. For example, the tissue imaging platform 120 may include the memory 230 that may include one or more programs 250 to perform one or more functions of the disclosed embodiments. For example, in some embodiments, the tissue imaging platform 120 may utilize one or more predictive model systems (e.g., deep learning models) to autonomously annotate an image of a tissue sample with a mapping estimate of cell types over each tissue region of the tissue sample. The one or more predictive model systems may be trained on the annotated training images stored on database 118 or locally on database 280. According to some embodiments, program 250 may include a machine learning model 290 that may be used to implement the one or more predictive model systems. According to some embodiments, machine learning model 290 may include a trained convolutional network, a trained recurrent neural network, a trained multilayer perceptron network, or combinations thereof.

The memory 230 may include one or more memory devices that store data and instructions used to perform one or more features of the disclosed embodiments. The memory 230 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational or non-relational databases. The memory 230 may include software components that, when executed by the processor 210, perform one or more processes consistent with the disclosed embodiments. In some embodiments, the memory 230 may include a database 280 for annotated training images for training and refining the machine learning model 290 used by tissuing imaging platform 120 to autonomously generate mapping estimates of cell types for each tissue region of a tissue sample.

The tissue imaging platform 120 may also be communicatively connected to one or more memory devices (e.g., databases) locally or through a network. The remote memory devices may be configured to store information and may be accessed and/or managed by the tissue imaging platform 120. By way of example, the remote memory devices may be document management systems, Microsoft™ SQL database, SharePoint™ databases, Oracle™ databases, Sybase™ databases, or other relational or non-relational databases. Systems and methods consistent with disclosed embodiments, however, are not limited to separate databases or even to the use of a database.

The tissue imaging platform 120 may also include one or more I/O devices 220 that may comprise one or more interfaces for receiving signals or input from devices and providing signals or output to one or more devices that allow data to be received and/or transmitted by the tissue imaging platform 120. For example, the tissue imaging platform 120 may include interface components, which may provide interfaces to one or more input devices, such as one or more keyboards, mouse devices, touch screens, track pads, trackballs, scroll wheels, digital cameras, microphones, sensors, and the like, that enable the tissue imaging platform 120 to perform aspects consistent with the disclosure.

In example embodiments of the disclosed technology, the tissue imaging platform 120 may include any number of hardware and/or software applications that are executed to facilitate any of the operations. The one or more I/O interfaces may be utilized to receive or collect data and/or user instructions from a wide variety of input devices. Received data may be processed by one or more computer processors as desired in various implementations of the disclosed technology and/or stored in one or more memory devices.

While the tissue imaging platform 120 has been described as one form for implementing the techniques described herein, other, functionally equivalent, techniques may be employed. For example, some or all of the functionality implemented via executable instructions may also be implemented using firmware and/or hardware devices such as application specific integrated circuits (ASICs), programmable logic arrays, state machines, etc. Furthermore, other implementations of the tissue imaging platform 120 may include a greater or lesser number of components than those illustrated.

FIG. 3 is a block diagram (with additional details) of the spatial transcriptomics platform 130, as also depicted in FIG. 1. According to some embodiments, spatial transcriptomics platform 130 may have a similar structure and components as those described with respect to tissue imaging platform 120 shown in FIG. 2. Thus, a full description of processor 310, I/O 320, memory 330, OS 340, program 350, database 360 is omitted for brevity. In addition to the above-referenced components, spatial transcriptomics platform 130 may include a sequencer 370. Sequencer 370 may include any DNA sequencing platform known in the art, including but not limited to, an Illumina sequencer, an ion torrent genexus system by ThermoFisher, and any other transcriptome sequencer known in the art.

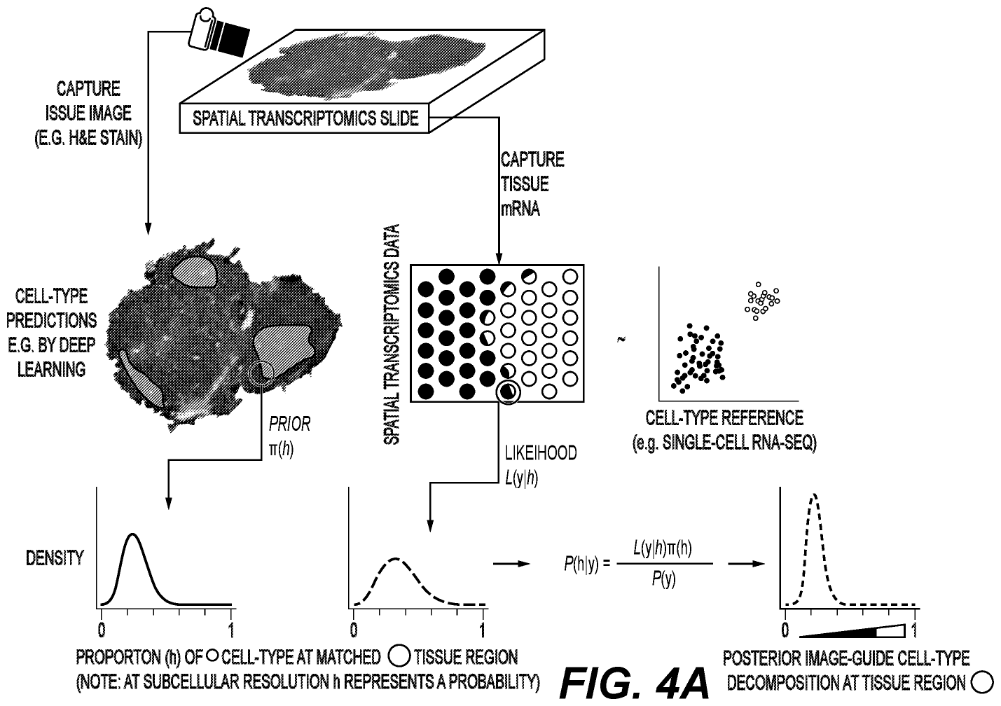

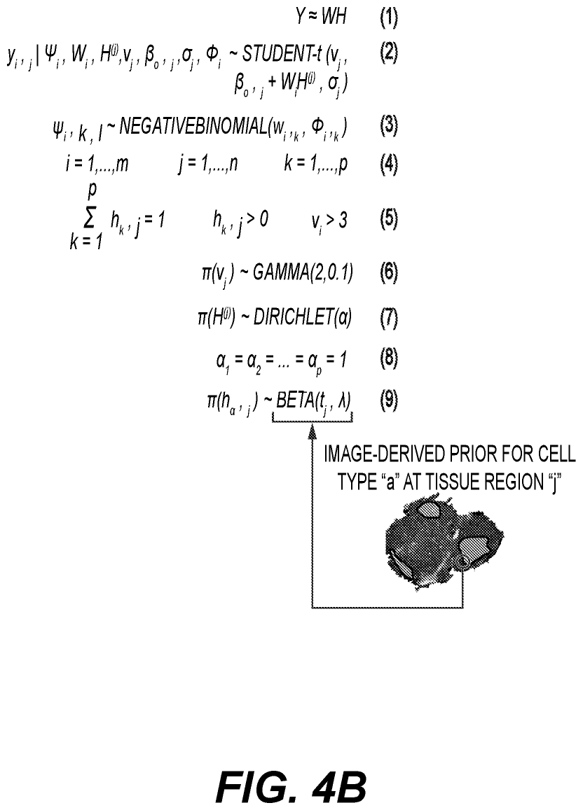

FIG. 4A provides a simplified schematic representation of GIST. The schematic shows a hypothetical tissue sample, where the location of a hypothetical cell-type (colored orange) is to be identified; this could represent, for example, immune cell infiltration in a tumor, although any cell types may be identified using the GIST system. Estimates of this cell-type from a deep learning model applied to a hematoxylin and eosin (H&E) stain image (left) are used to optimize the estimates derived from the spatial transcriptomics data (right), yielding improved estimates over what could be achieved from either approach alone (bottom right). It should be noted this technique is not limited to an H&E stain image, and similar results may be accomplished by using other pathological imaging techniques, including but not limited to immunostaining, for example immunohistochemical staining and immunofluorescence staining, hybridization probes, including fluorescence in situ hybridization (FISH), and staining with tissue marking dyes such as H&E and other tissue marking dyes known in the art. As shown in FIG. 4A the methodology starts with a tissue sample prepared on a spatial transcriptomics slide. Image data is collected, for example, by applying an H&E stain to one side of the tissue sample, and capturing image data of the stained tissue sample. The stained tissue sample may then be transmitted to tissue imaging platform 120, which is a computer-based system trained with pathologist annotated medical images to detect cell types based on the captured image data. The tissue imaging platform 120 may process the stained tissue sample image data and output a mapping estimate of cell types for each tissue region of the tissue sample. Tissue imaging platform 120 may utilize one or more deep learning models (e.g., machine learning model(s) 295), including but not limited to a convolutional neural network trained on annotated pathology images, a recurrent neural network trained on annotated pathology images, a multilayer perceptron network trained on annotated pathology issues, or any other machine learning model known in the art. Simultaneously, the spatial transcriptomics slide allows for an operator of system 100 to permeabilize the cells of the tissue sample and apply primers to the nucleic acids (e.g., RNA) located within the cells of the tissue sample. The primers include a spatial barcode that uniquely spatially identifies each spot on the spatial transcriptomics slide. The tissue sample that is attached to the spatial transcriptomics slide acts as a template for a reverse transcription reaction, which may generate a complementary nucleic acid (e.g., DNA) library. The complementary nucleic acids may be cleaved from the spatial transcriptomics slide and collected for sequencing. The collected complementary nucleic acids may be provided to the sequencer (e.g., sequencer 370) of the spatial transcriptomics platform 130. The mapping estimate produced by tissue imaging platform 120 may be used as a Bayesian prior on the output of the spatial transcriptomics data generated by the spatial transcriptomics platform 130, and an exemplary mathematical model for implementing the GIST methodology is described in more detail with respect to FIG. 4B.

FIG. 4B provides a mathematical notation describing an example GIST model: the spatial transcriptomics data generated by spatial transcriptomics platform 130 can be represented by a matrix Y_{m×n}, where m and n represent rows and columns, respectively, of matrix Y. Rows m represent each gene present within the given tissue sample, and columns n represent n “spots” (e.g., tissue regions) of the tissue sample. Accordingly, the values within each element of matrix Y represents the proportion of gene m expression in tissue region n. The GIST model provides that the spatial transcriptomic data can be approximately factorized as a cell-type signature matrix W_{m×p} and a matrix of cell-type compositional estimates H_{p×n} (equation (1)). The rows of matrix W, represented by m, represent each gene present within the given tissue sample, and columns p of matrix W represent each cell type present within the tissue sample. The rows of matrix H similarly represent each cell type present within the tissue sample while columns n represent n tissue regions of the tissue sample. The GIST model provides that m genes are indexed by i, n tissue regions are indexed by j, and p cell types are indexed by k. The matrix factorization described above is represented by equation (1). The GIST model provides for estimating H, which can be understood as an inferred cell type (e.g., k) compositional map for each tissue region (e.g., j) of the tissue sample by using the model mathematically described by equations (2-9).

Cell-type composition H can be estimated using the model in equations (2)-(9). A single cell RNA-seq dataset from the same tumor type is represented by Ψ. The single-cell RNA-seq data may be collected directly from the spatial transcriptomics slide, or it may come from a different tissue section that includes the same types of cells as the tissue of the spatial transcriptomics slide. For example, the single-cell RNA-seq data may be generated using an adjacent tissue section. It should be noted that in some embodiments, values of W may be inferred directly from the spatial transcriptomics data, represented by Y. In embodiments in which W is inferred from values of Y, the GIST methodology may include utilizing a latent variable model to infer the values of W directly from the values of the spatial transcriptomics data. In some embodiments, the GIST methodology may include inferring the values of W from Y using a latent Dirichlet allocation model.

Each element of W may be estimated from Ψ using a negative binomial distribution (with overdispersion parameter ϕ_{i,k}) estimated for each gene i, in each cell type k, from the expression in each single-cell l. The equation for estimating values of Ψ is shown below as the following:

$${Ψ}_{i , k , l} ∼ NegativeBinomial ( {w}_{i , k} , {ϕ}_{i , k} ) , i = 1 , … , m ; k = 1 , … , p .$$

The following regression model may be used for estimating values of H:

$${y}_{i , j} \vert {Ψ}_{i} , {W}_{i} , {H}^{( j )} , {v}_{j} , {β}_{0 , j} , {σ}_{j} ∼ Student - t ( {v}_{j} , {β}_{0 , j} + {W}_{i} {H}^{( j )} , {σ}_{j} ) i = 1 , … , m ; j = 1 , … , n ; k = 1 , … , p .$$

Equation (5) shows the model constraints:

$$\sum {h}_{k , j} = 1 , {h}_{k , j} > 0$$

Equations (6)-(9) show the priors, denoted by π. For example, a gamma prior on the degrees of freedom of the t-distribution and a Dirichlet prior on the columns of the H matrix may be used as shown below:

$$\begin{matrix}π ( {v}_{j} ) ∼ Gamma ( 2 , 0.1 ) \\ π ( {H}^{( j )} ) ∼ D i r i c h l e t ( α ) ; {α}_{1} = {α}_{2} = … = {α}_{p} = 1\end{matrix}$$

Other parameters are assigned weakly informative priors. The key informative prior is shown in equation (9), where the image-derived prior estimate of cell type composition for a cell type of interest, contained in row a of H, is specified as a beta distribution as shown below:

$$π ( {h}_{k = c , j} ) ∼ Beta ( {τ}_{j} , λ )$$

As shown in the equation above, τⱼ is the mean of the beta distribution representing image-derived value for the proportion estimate of cell-type c capturing our prior belief. λ is a hyperparameter, representing the total count parameter of beta distribution, determining how much weight is to be placed on the image data and how much to place on the transcriptomic data. As shown above, τⱼ specifies the prior cell type composition estimate from the image, and the concentration parameter λ, a scalar, determines how much weight to place on the image data and how much to place on the transcriptomic data. The superscript notation (e.g. H^{(j)}) denotes the columns of a matrix. Vectors are shown using boldface and matrices bold capital letters. All equations herein assume m genes (indexed by i), n tissue regions (e.g. spots, indexed by j), p cell types (indexed by k). It should be noted however, that in some embodiments the image-derived prior estimate of cell type composition may be specified as a Dirichlet distribution, a normal distribution, or any other parameterized probability distributions known in the art.

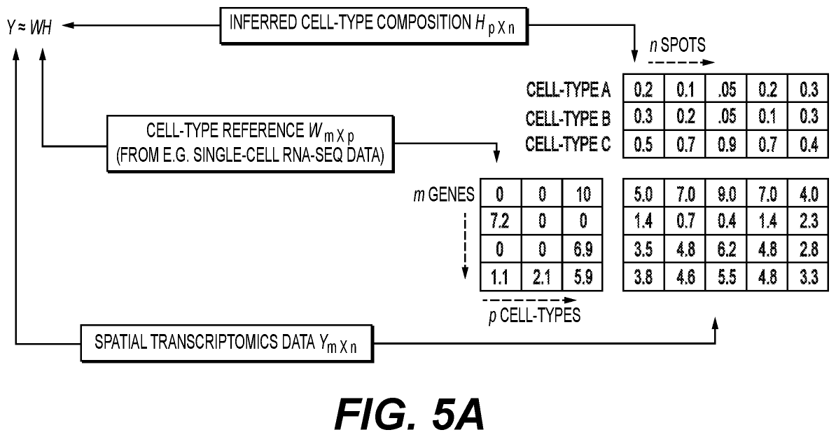

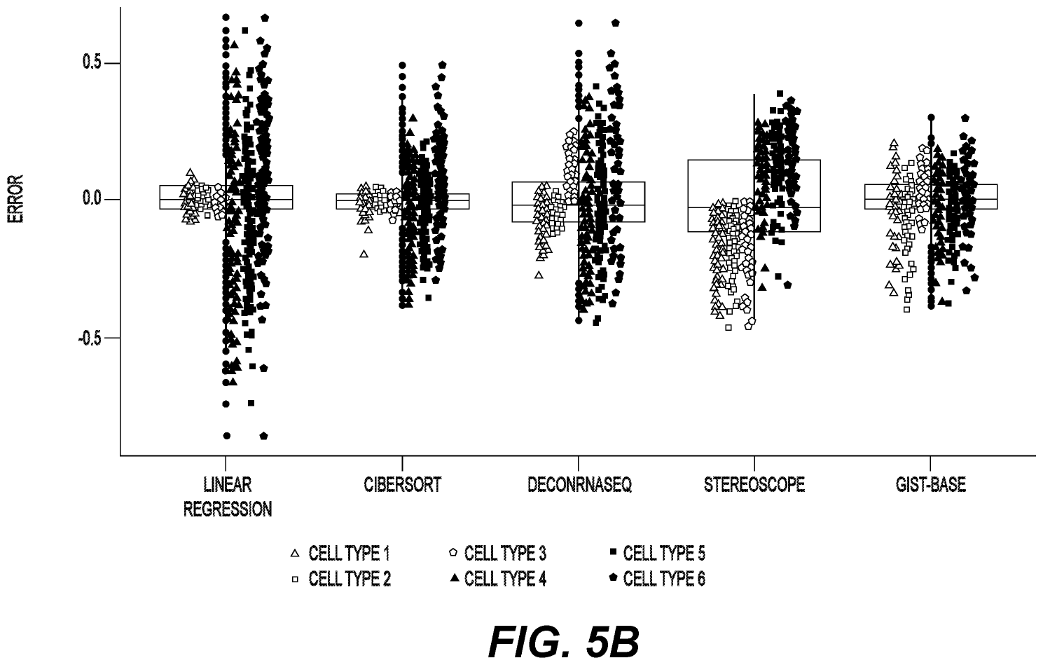

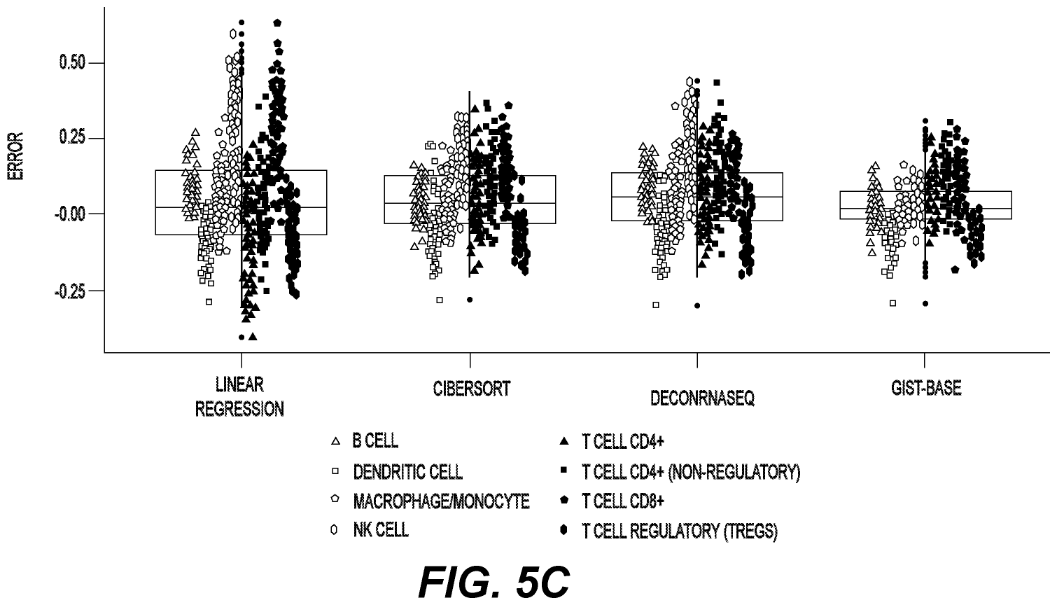

FIG. 5A shows spatial transcriptomics expression data is arranged in an m genes by n spots matrix Y. As described above with respect to FIGS. 4A-4B, Y can be decomposed into a basis matrix W and a matrix H that contains the proportion of each of p cell-types on each spot or (at subcellular resolution) the probability that a spot matches a cell-type (shown for three hypothetical cell-types A, B and C). The basis matrix W is typically known and can be derived for example from single-cell RNA-seq data from the same or similar tissue.

FIG. 5B shows the performance of the GIST base-model (e.g., GIST model before applying the Bayesian priors determined from the image data mapping estimates to the spatial transcriptomic data) compared to some existing approaches, including a linear regression model, CIBERSORT, DeconRNAseq, and Stereoscope. CIBERSORT and DeconRNAseq are methods originally designed for bulk expression data (e.g., non-spatially resolved transcriptomic data obtained from a homogenized bulk mixture of cells of a tissue sample). Stereoscope is a method tailored specifically for spatial transcriptomics data, and linear regression is the simplest applicable model. A mixture of 6 cell types was simulated using the tool splatter. Points have been colored by the simulated cell type and the y-axis shows the deviation from ground truth, quantified by the difference between the estimated cell type proportions in a sample and the true proportion used for the simulation. The Mean Absolute Distance, summarizing the overall performance of each method is as follows (lower values imply better performance): Linear regression=0.13, CIBERSORT=6.8×10⁻², DeconRNAseq=0.11, Stereoscope=0.15, GIST-base model=7.4×10⁻². Accordingly, it is clear that the GIST methodology performs competitively to existing methods in determining cell type composition of a tissue sample even before applying priors obtained from image data. While CIBERSORT performed slightly better on the splatter simulations than the GIST base-model, the GIST base-model outperformed CIBERSORT on the other benchmarking dataset shown in FIG. 5C.

FIG. 5C shows simulated dataset obtained from a benchmarking procedure outlined in Strum et al. Points have been colored by the immune cell type and the y-axis shows the deviation from ground truth, quantified by the difference between the estimated cell type proportions in a sample and the true proportion used for the simulation. The Mean Absolute Distance, summarizing the overall performance of each method is as follows (lower values imply better performance): Linear regression=0.14, CIBERSORT=0.09, DeconRNAseq=0.1, GIST-base model=6.4×10⁻². Accordingly, the GIST-base model substantially outperformed other methods in this simulation even before applying priors obtained from image data.

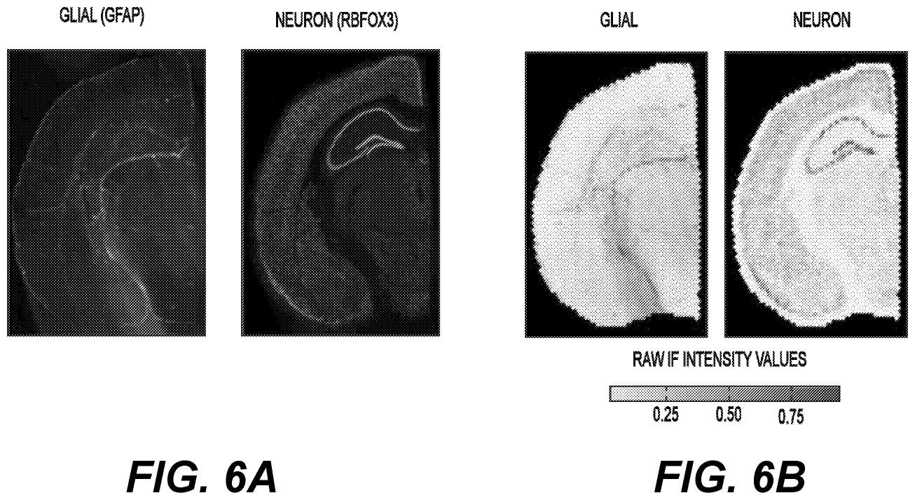

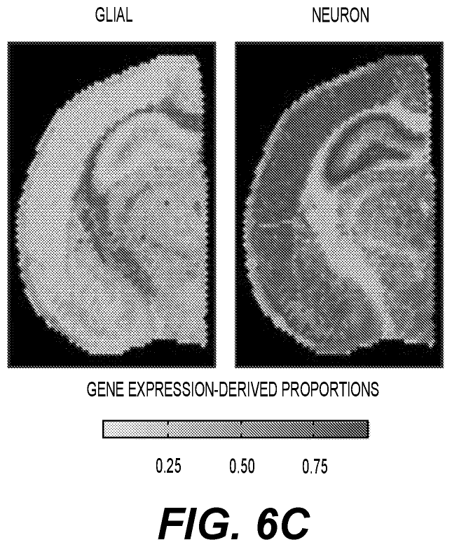

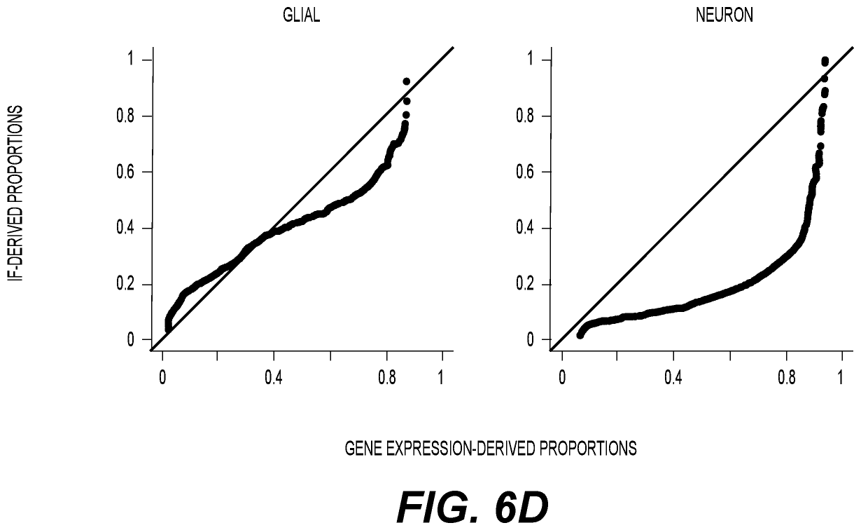

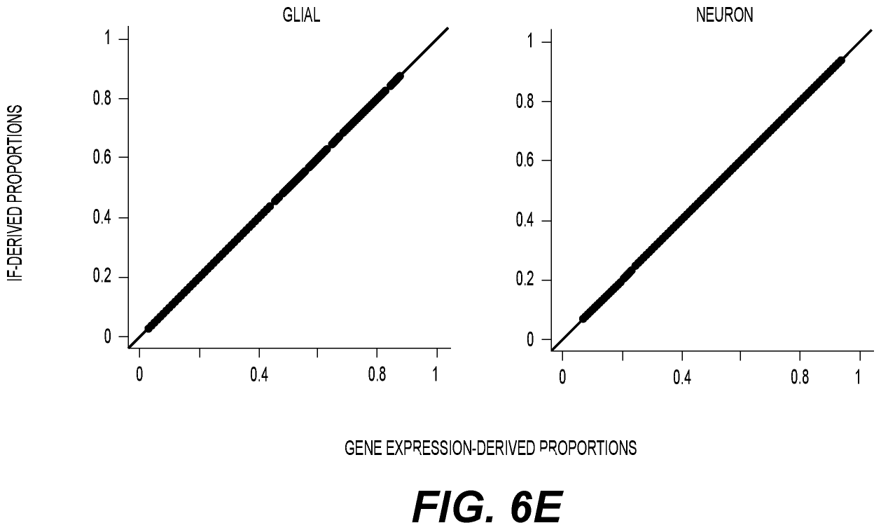

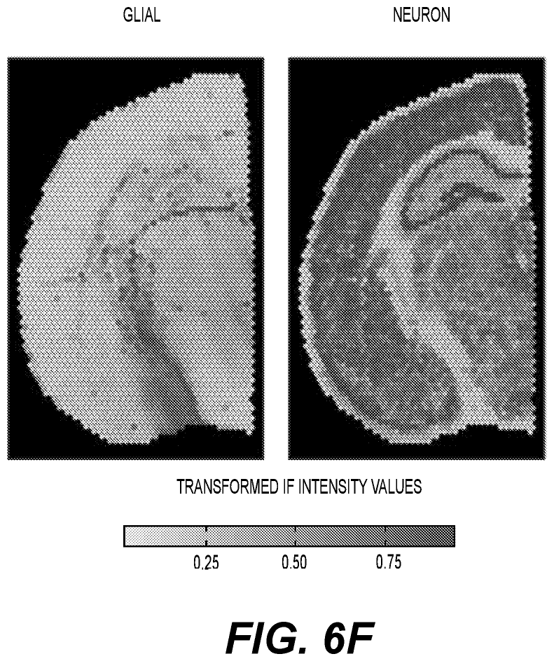

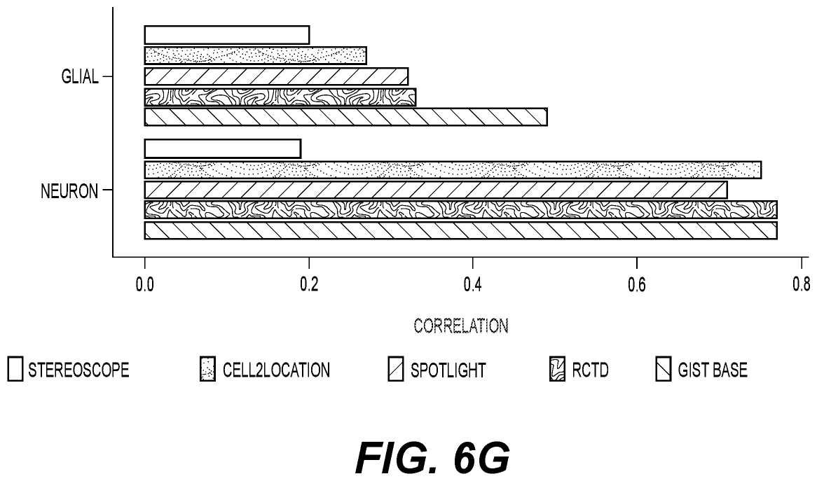

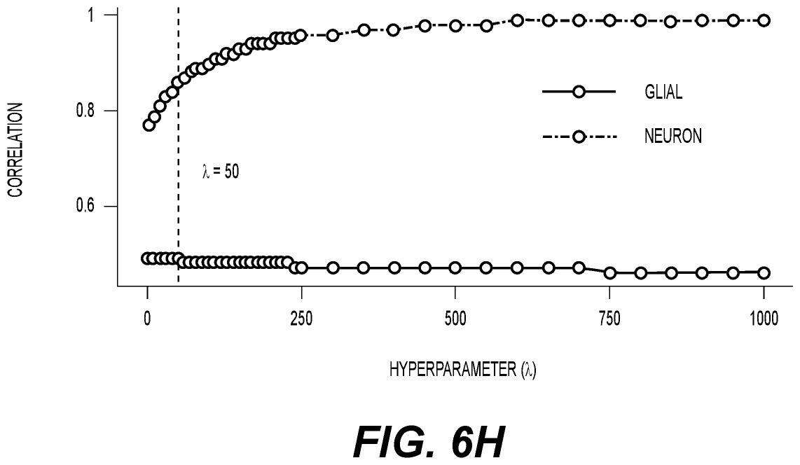

FIG. 6A shows an immunofluorescence image of mouse brain tissue section with glial (Gfap) and neuronal (Rbfox3) cell markers. After verifying that the GIST-base model performs competitively on simulated data when compared to existing methods, a publicly available dataset was utilized that measured gene expression in the mouse brain using the 10× Visium spatial transcriptomics platform. In addition, an immunofluorescence (IF) stain was applied on the reverse side of the tissue section. IF staining was performed for two proteins, Gfap and Rbfox1, which are cell markers that are unique to glia and neurons, respectively. FIG. 6B is a spatial distribution of raw IF intensity values for the two channels for glial and neuronal markers averaged over overlapping spatial transcriptomics spots. Intensity values are rescaled from 0 to 1 before being averaged over a spot. The spot-level intensity estimates shown in FIG. 6B were used to represent an independent ground-truth that approximates the abundance of neurons and glia in the regions of the slide overlapping each of 4,992 Visium spots (e.g., tissue regions).

FIG. 6C is a spatial distribution of aggregate glial and neuronal proportions estimated using the spatial transcriptomics gene expression data alone. All proportions of subtypes belonging to the glial cell type were summed up to arrive at the aggregated value. A similar procedure was used for the neuronal cell type.

FIG. 6D shows a quantile-quantile plot (QQ plot) of image-based IF-derived values for total glial and neuronal content for a spot (y-axis) versus values obtained for total glial and neuronal content from the spatial transcriptomics gene expression data (x-axis). The reference dataset (FIG. 6B) was used as a reference dataset to compare the performance of the GIST base-model, Stereoscope, RCTD, Cell2location and Spotlight against the IF derived ground truth. However, as can be seen in FIG. 6D, the image-derived and spatial transcriptomics derived cell proportion estimates followed different distributions, so the IF derived estimates were normalized by mapping them onto quantiles of the spatial transcriptomics derived estimates, the results of which are shown in FIGS. 6E-6F.

FIG. 6E shows a QQ plot of image-based IF-derived values for glial and neuronal content for a spot (y-axis) versus values obtained for total glial and neuronal content from gene expression data (x-axis). The QQ plot of FIG. 6E is generated after a post-mapping strategy where the distribution of cell type content estimated from image-based strategy is mapped on to the distribution of cell type content estimated from the spatial transcriptomics gene expression data. FIG. 6F provides a spatial distribution of IF intensity values for the glial and neuronal channel where the values have now been mapped to a distribution estimated from the gene expression data.

FIG. 6G shows a bar plot showing Spearman correlation between the total cell type content and IF-based ground truth (x-axis) for five different gene expression-based cell type decomposition methods. As shown in FIG. 6G, the GIST base-model had slightly higher performance than the other methods (RCTD, Cell2location, Stereoscope, RCTD). The GIST-base model achieved a Spearman's rank correlation of 0.49 and 0.77 for glial and neuronal cells, respectively, compared to 0.33 and 0.77 for RCTD, the second best performing method. Accordingly, the results show that the GIST base-model performs competitively compared to existing methods for cell-type decomposition in real spatial transcriptomics data.

Although the GIST base-model performed well compared to existing computational methods, the results also showed that even the best performing methods for spatial transcriptomics cell-type decomposition (e.g., GIST-base and RCTD) were not markedly different in performance from each other and neither achieve an optimal level of performance when compared to the IF derived ground-truth. In order to achieve a higher level of performance, image-derived prior information was utilized in the Bayesian model described with respect to FIGS. 4A-4B to enhance the GIST-base model. Using the Rbfox1 IF data, an image-derived prior was derived, which provides prior evidence of the abundance of neuronal cell-types over each spatial transcriptomics spot (e.g., tissue region of the tissue sample). The priors were specified using a beta distribution, and was applied to the group of model parameters that estimate neuronal cell types. The beta distribution was parameterized by the mode (ϕ; the point estimate of the cell-type from the image) and the concentration parameter (λ; the strength of the prior, corresponding to the weight placed on the image). The beta distribution is naturally constrained to a 0-1 scale, which makes it appropriate for specifying prior estimates of cell-type composition. A key question that was empirically resolved was how much weight to place on the image-derived priors and how much weight to place on the spatial transcriptomics data itself. This is determined by tuning the hyperparameter λ. Too small of a value of λ will mean there is little to no influence of the image derived prior cell-type on the model, but selected a value too large for λ will overfit the model to the image and degrade performance.

FIG. 6H shows a line plot showing change in performance measured as Spearman correlation with IF derived ground truth (y-axis) versus the prior hyperparameter λ (x-axis). The image-based prior is only applied to the neuronal cell type. The vertical dashed red line indicates a stopping point (λ=50) where performance in the glial channel began to deteriorate. Intuitively, this value of λ concentrates most of the prior probability weight within +/−10% of the mode. Accordingly, a λ value of 50 was used for the remaining tests.

FIG. 6I provides scatter plots comparing the cell type decomposition estimates derived from the spatial transcriptomics gene expression data when (top row) the GIST-base model is applied and no prior information is leverage or (bottom row) when IF-derived cell type compositional estimates are incorporated as prior information in the (left column) glial and (right panel) neuronal cell types with using a λ value of 50. Notably, at this λ value, the Spearman's rank correlation between the GIST model derived neuronal cell-type estimates and the IF derived ground truth increased from 0.7 to 0.85, which is substantially better than any method that does not leverage the images, and also approaches optimal performance. These results support the notion that applying informative prior information derived from matched images has the potential to greatly improve performance of cell-type decomposition in spatial transcriptomics data and additionally provides a reasonable estimate of the key hyperparameter λ that may be applied to additional datasets. However, while IF stains very reliable markers, they are restricted to a small number of protein targets and are much less commonly collected than the H&E stain, which is considered to be the standard pathology stain collected as part of almost all spatial transcriptomics protocols. Accordingly, it was an objective of the GIST model to show that image-information derived from deep learning models applied to H&E stains can be used to improve cell type decomposition in spatial transcriptomics data. Deep learning models (e.g., convolutional neural nets) have already been developed that can output numerous classes of annotations from H&E stain tissue sections alone, such as cell-type information, ploidy, and immune cell infiltration.

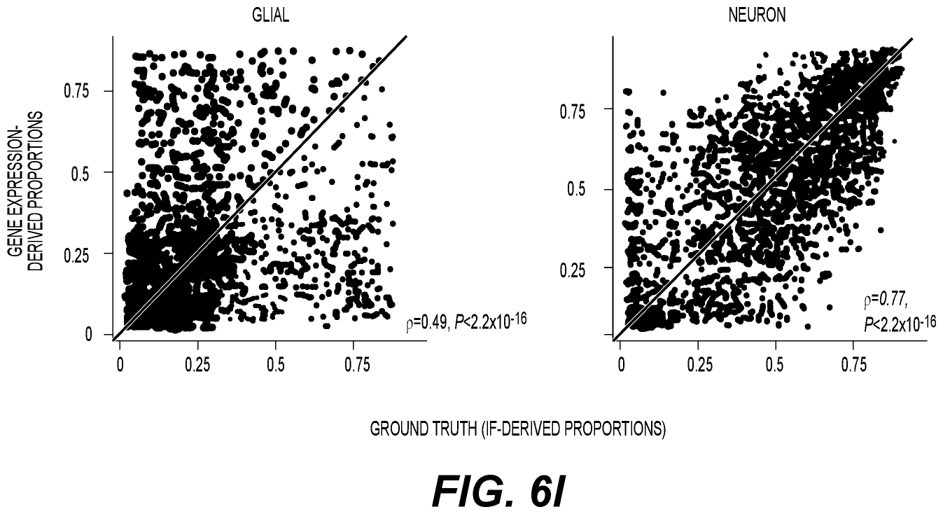

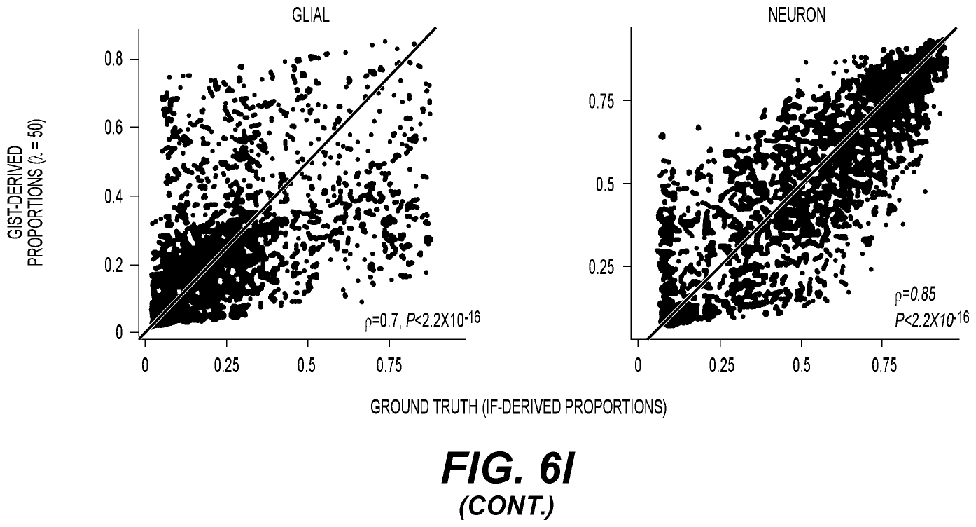

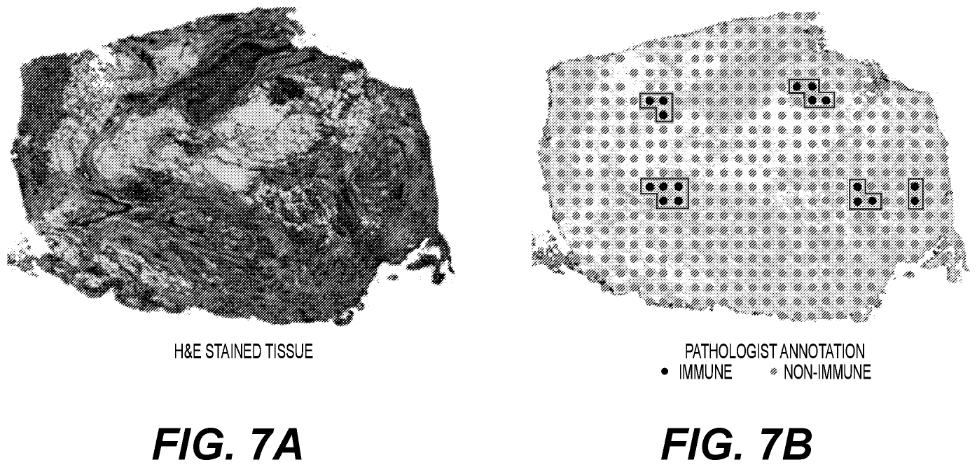

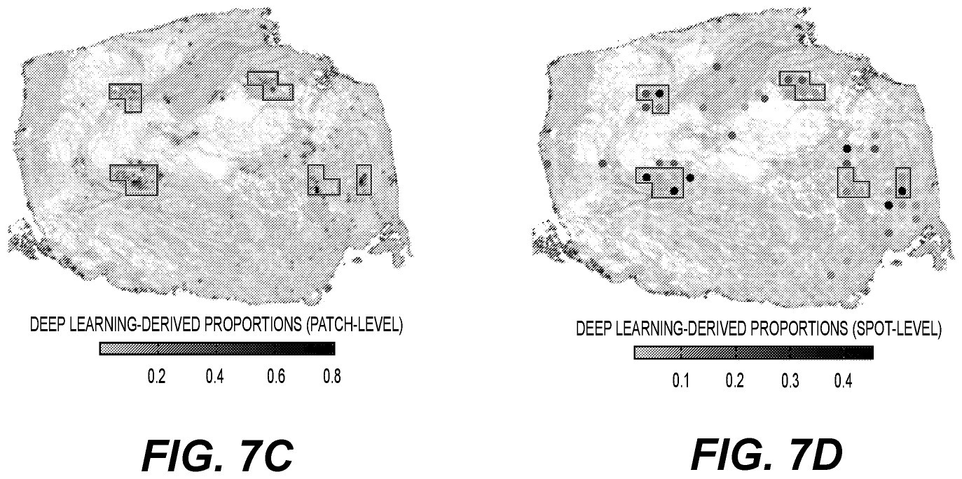

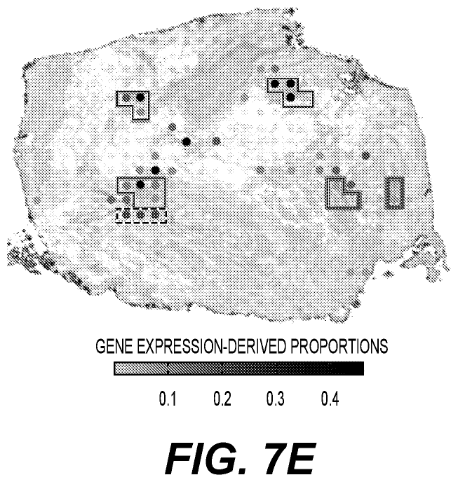

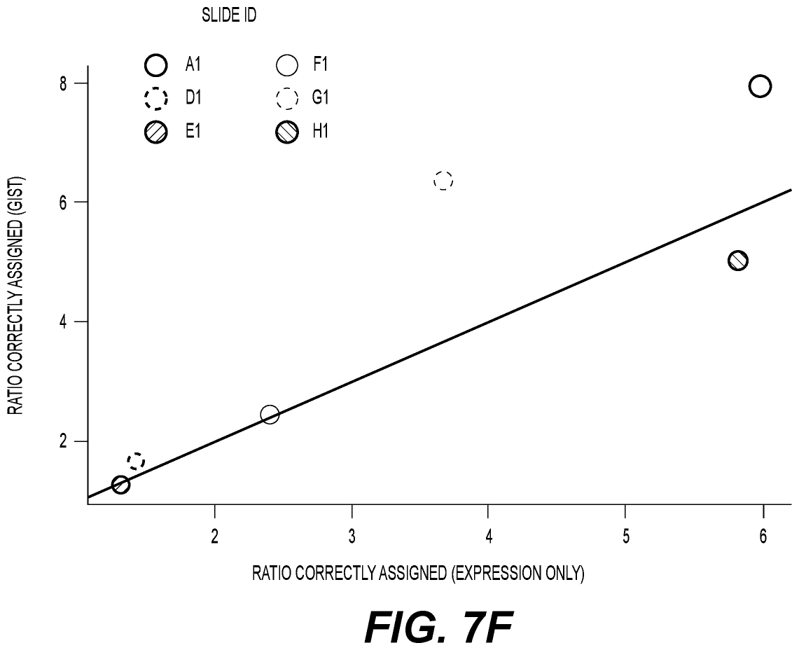

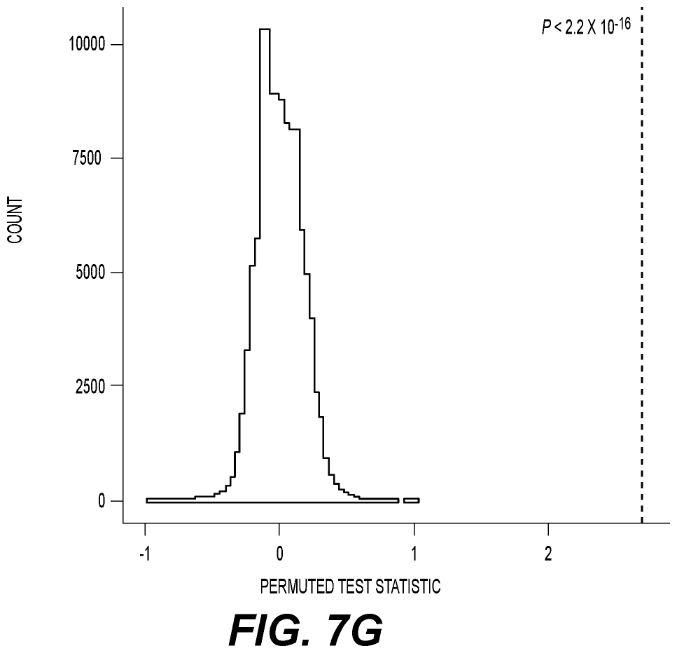

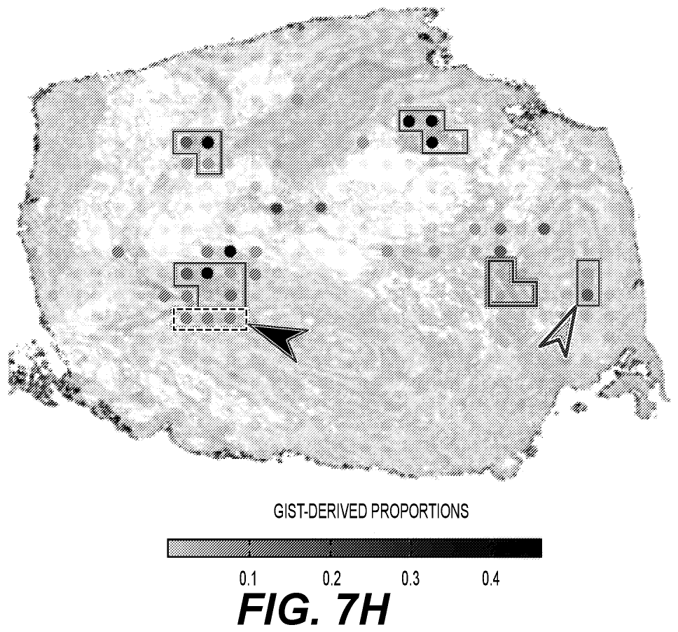

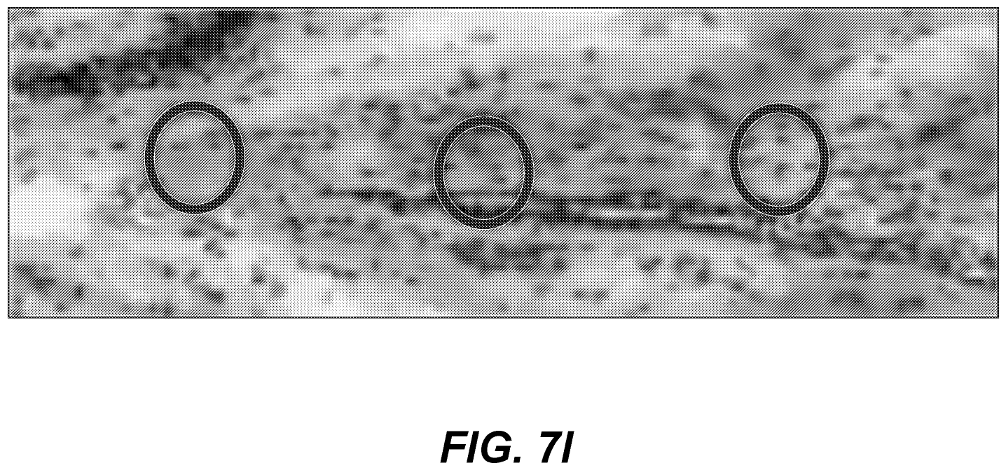

Accordingly, tests were performed on 8 previously published spatial transcriptomics slide tissues, which had measured gene expression in biologically independent breast cancer tumors, the analysis of which is shown in FIGS. 7A-7I. Notably, each of these tissue sections had also been H&E stained, and regions of immune cell infiltrate had been annotated by a pathologist, which provided an independent ground truth against which to assess the GIST model's performance. A deep learning model, specifically a convolutional neural network, that was trained to identify regions of tumor infiltrating lymphocytes from H&E stained tissue section images was used to generate image prior data. It should be noted that any other deep learning approach, such a recurrent neural network, a multilayer perceptron network, etc. may have been used in lieu of a convolutional neural network to provide image prior data to the GIST model. FIG. 7A shows an example H&E stained tissue image obtained from the reverse side of the breast cancer spatial transcriptomics slide G1. FIG. 7B provides a pathologist annotation for slide G1 showing regions containing spatial transcriptomics spots that were labelled immune cell infiltrated (marked by dark colored spots and green outlines). As discussed above, the pathologist annotation was used as an independent ground truth to assess model performance. FIG. 7C provides output from the deep learning model for slide G1 plotted on top of the breast cancer tissue section. The color scale indicates deep learning-derived predictions for the proportions of immune cells made on 50×50 micron patches of the tissue. Green boxes outline regions of pathologist's annotated immune spots. The deep learning derived predictions were averaged over the pixels overlapping each of the spatial transcriptomics slide spots, which yielded a deep-learning derived per-spot estimate of immune cell levels (in an approach similar to the one applied for IF data, described above with respect to FIGS. 6A-6I), as provided in FIG. 7D. The spot level predictions shown in FIG. 7D can be used as priors in the GIST model. Spot level predictions are a sum of patch level predictions weighted by their percent overlap with the spot. Boxes outline regions of pathologist's annotated immune spots.

FIG. 7E provides gene expression-derived immune cell proportions from the GIST base-model. Solid boxes indicate regions of pathologist's annotated immune spots. Green indicates that the model reasonably identifies immune-infiltrated spots and red indicates that the immune spots were not captured. The dashed black box indicates a region of interest that likely is a false positive (see FIGS. 7H and 7I). Then, a similar normalization approach as was described for the IF data (mapping the deep learning derived estimates to the quantiles of the gene expression derived estimates) was applied to these deep-learning derived immune cell compositional estimates as Bayesian priors (as described with respect to FIGS. 4A-4B and equations (1) to (9)) which were specified as a beta distribution on the appropriate GIST model parameters. A λ value of 50 was used based on the empirically determined optimal value determined with respect to FIG. 6H. When the model is performing well, it should identify more immune cells in pathologist-annotated immune cell regions, but less in other regions of the slides. Accordingly, model performance was estimated based on the ratio of immune cells identified within the pathologist annotated regions of immune infiltrate compared to all other regions of the tissue. FIG. 7F provides a scatterplot showing performance of GIST model (y-axis) versus the performance of a base-model based on only gene expression data (x-axis) for six pathologist annotated spatial transcriptomics slides. Performance is defined as the ratio of the median proportion of immune cells in pathologist labeled immune cell slide spots, versus the median proportion of immune cells in the other slide spots. Points are colored by slide. The red line is the identity line. As can be seen in FIG. 7F, performance increases were observed in four out of the six slides and was substantial in two out of the six slides, as can be seen in FIG. 7G, which shows a 1.95 and 2.69 times performance increase for slides A1 and G1, respectively. FIG. 7G is a histogram showing empirical null distribution of ratio-based test statistic generated using a permutation procedure (x-axis). The test statistic is a measure of improvement in model performance, versus the pathologist-annotated ground truth, when deep-learning derived prior cell type annotations are incorporated. The observed test statistic is shown using a vertical red line.

FIG. 7H provides GIST-derived immune cell proportions when the deep learning cell-type annotation have been used to improve the model. Solid boxes indicate regions of pathologist's annotated immune spots. Green indicates that immune spots are successfully identified, and red indicates that immune spots were not well captured. The dashed black box, highlighted by the black arrow, indicates the same region of interest as in FIG. 7E, where the false positive immune cell predictions have been mitigated. The green arrow highlights a region where the correct identification of a pathologist annotated immune-infiltrated regions has improved. Thus, it can be seen that leveraging deep learning derived prior information has the potential to improve cell-type decomposition in spatial transcriptomics data in the GIST model. FIG. 7I shows tissue section overlapping the region of interest shown in FIGS. 7E and 7H showing the spatial transcriptomics spots. The H&E stain shows minimal evidence of immune infiltration. Accordingly, the image derived prior was able to perform predictive performance in the tissue region identified in FIG. 7I.

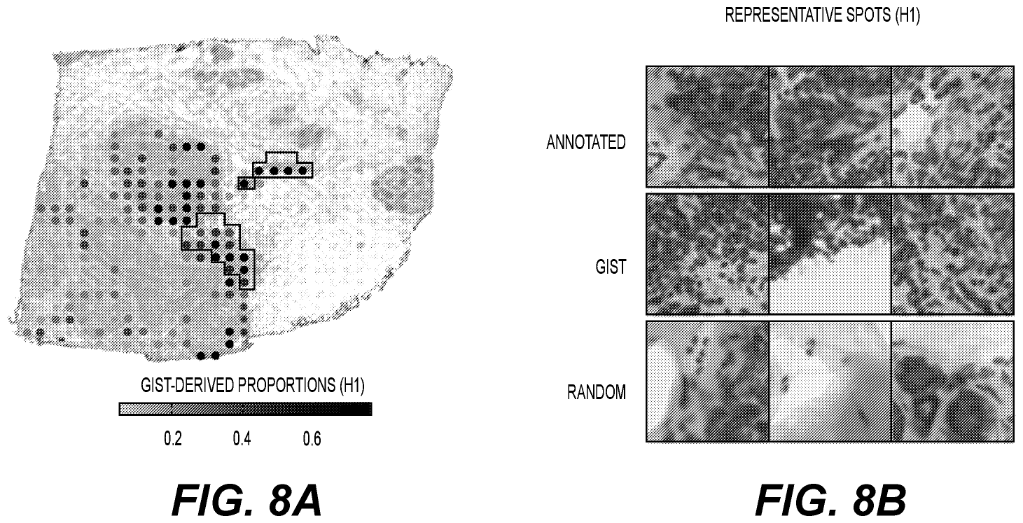

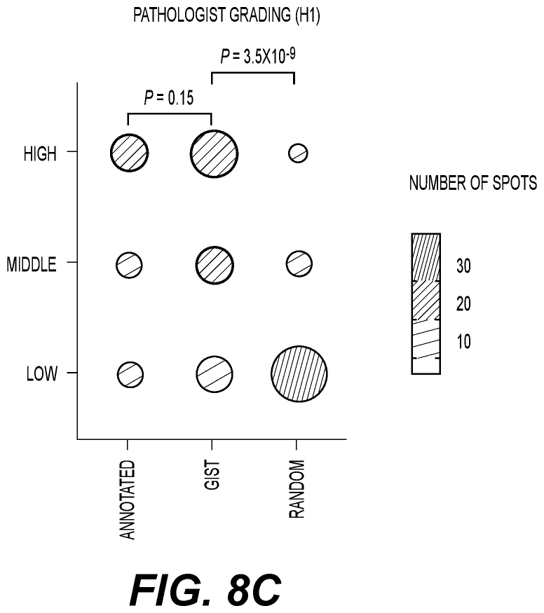

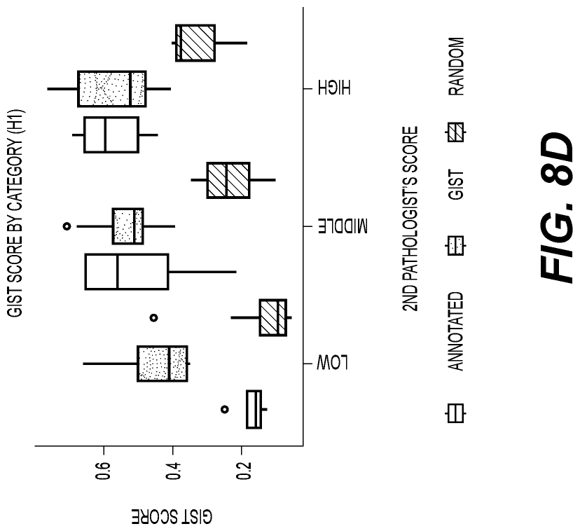

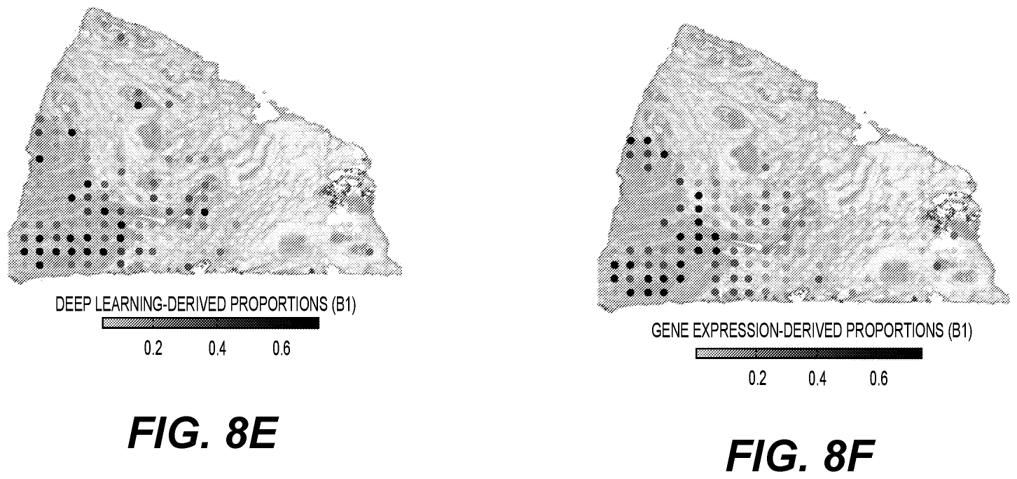

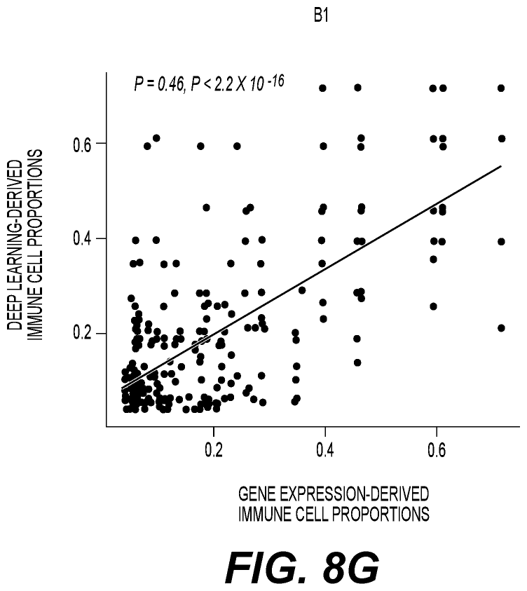

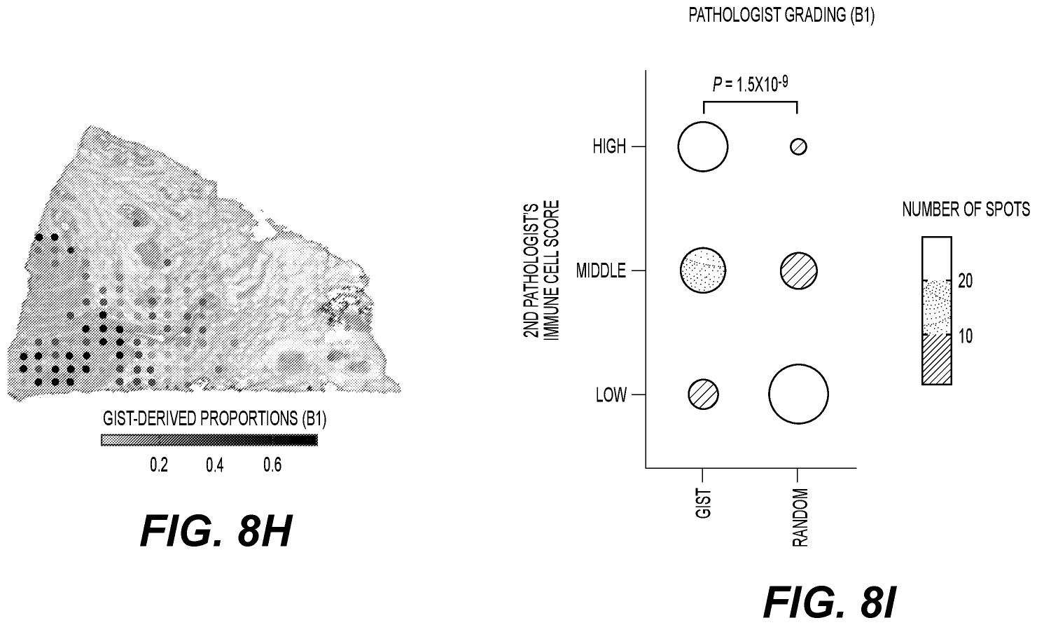

Not only does the GIST model improve performance as compared to other computational methods, the GIST model also can lead to better-than-pathologist performance in cell-type annotation. As shown in FIG. 7F, slide H1 exhibited a decrease in performance when the GIST-base model was enhanced with the image derived prior data. However, upon closer inspection it became clear that there was a large region of the tumor that was identified as immune cell infiltrated by both the spatial transcriptomics assay and the deep learning mode, but this region was not marked by the initial pathologist annotation. Thus, this region was predicted as heavily immune cell infiltrated by the GIST model in large regions outside of those annotated as immune infiltrated by the initial pathologist. FIG. 8A provides an example GIST model-derived proportions plotted on top of tissue from slide H1. Green outlines indicate original annotation of immune spots identified by first pathologist. It was hypothesized that the region outside of the green outlines represented an error in the initial pathologist's annotation rather than a deficiency in the GIST model prediction. To test this, a second independent pathologist was tasked with re-examining the relevant regions of the slide, while remaining blinded to the GIST model's output and to the original annotation by the initial pathologist. The second pathologist was present with small subregions from slide H1 and asked to categorize them as either low, middle, or high levels of immune cell infiltrate. These regions were chosen from either (i) the first pathologist's annotated immune cell regions, (ii) high-confidence immune cell regions identified by the GIST model but not the first pathologist, or (iii) other randomly chosen regions. This experiment is represented in FIG. 8B, which shows three representative 100×100 micron images enclosing spots each from first pathologist's annotated region (top), other high confidence regions from GIST model (middle) and randomly selected regions (bottom). Spots are taken from slide H1. The second pathologist's reannotation determined no statistical difference between the high-confidence regions or immune cell infiltrate annotated by the first pathologist, and the high-confidence regions identified by the GIST model, which were missed by the first pathologist, as shown in FIG. 8C, which provides a dot plot showing the second pathologist's immune infiltration grading with a score of low, middle and high (y-axis) for spots from different regions of the tissue (x-axis). Spots were taken from slide H1 from regions previously annotated by first pathologist as immune rich, high confidence regions from GIST model and random regions on the slide. It is notable that the high-confidence regions of immune cell infiltrate that were identified by GIST were much more likely to be marked as high probability regions of immune cell infiltrate compared to randomly chosen slide regions upon the second pathologist's reannotation, as can be seen in FIG. 8D. FIG. 8D is a boxplot showing distribution of GIST model predicted immune cell proportions (y-axis) broken down by immune infiltration grade (x-axis) provided by the second pathologist. For each pathologist grade (low, middle & high), GIST scores are shown for spots from annotated, GIST high confidence and random regions. Spots taken from slide H1. This result strongly suggests that the apparent poor performance on slide H1 arose from oversight in the original pathologist's annotation, not from a misidentification of the GIST model, and additionally shows that the GIST model is capable of identifying histopathological features that are missed by a human pathologist's manual annotation.

The two spatial transcriptomics slides where the original pathologist's annotation had not identified any regions of immune cell infiltration were also reexamined. FIG. 8E shows deep learning-derived proportions for spots on slide B1. The color scale shows predicted proportion of immune cells at a spot. Again, both the deep learning model and the expression-based cell-type predictions from the spatial transcriptomics assay (as shown in FIG. 8F) identified regions of immune cells, and similar slide regions were identified by both approaches (as shown in FIG. 8G). FIG. 8F shows gene expression-derived proportions for slide B1 from GIST base model. The color scale shows the predicted proportion of immune cells at a spot. FIG. 8G is a scatter plot showing per-spot correlation between deep learning-derived predictions (y-axis) and gene expression-derived proportions (x-axis) for slide B1. Each dot is a spot and the red line is the regression line. The results of FIG. 8G show that similar slide regions were identified by both the deep-learning and spatial transcriptomics approach, with a Spearman's correlation=0.46. These regions of slide B1 were identified by the GIST model in as shown in FIG. 8H (noting that the color scale shows the predicted proportion of immune cells at a spot or tissue region), thus it was again hypothesized that the original pathologist may have missed these immune infiltrated regions in the initial annotation of these slides. The same scoring procedure outlined above was used to reannotate the slides by the second pathologist. Again, the second pathologist's annotations agreed with those predicted by the GIST model, as seen in FIG. 8I, which is a dot plot showing the second pathologist's immune infiltration grading with a score of low, middle and high (y-axis) for spots from different regions of the tissue (x-axis). Spots were taken from slide B1 from high confidence regions from GIST model and random regions on the slide. The results seen in FIG. 8I strongly suggest that the GIST model had again correctly identified regions of immune cell infiltration that were missed by the first pathologist, which suggest that utilizing the GIST model that jointly leverages both deep learning predictions with spatial transcriptomics data has the potential to outperform not only traditional spatial transcriptomics methods, but also human pathologists in identifying these important tumor features. It should be noted that the GIST methodology outlined above is exemplary in nature, and could be applied to identify many other cell features within tissues, not limited to but including cell type composition in tissue samples tracking developmental processes, early disease detection and tracking, environmental impacts on tissue health and development, chromosomal ploidy, segmental copy number alternations, signaling pathway activity, disease subtype, gene expression signatures, cell proliferation status, gene expression programs, etc.

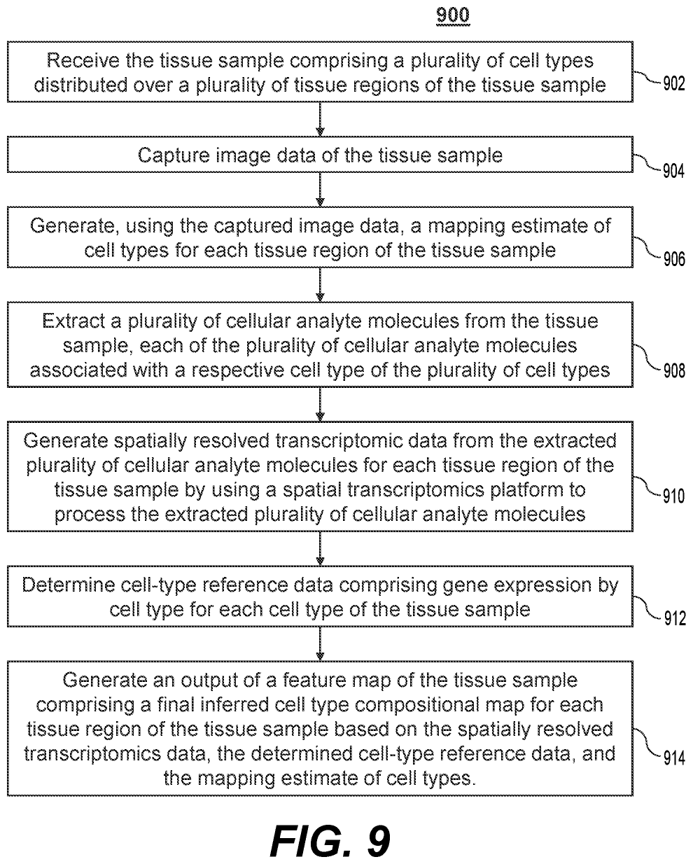

FIG. 9 is a flowchart showing an exemplary method 900 of generating a final inferred cell type compositional map using the GIST model, in accordance with an exemplary embodiment of the present invention. In step 902 of method 900, the method may include receiving a tissue sample that includes a plurality of cell types distributed over a plurality of tissue regions of the tissue sample. In step 904, the method may include capturing image data of the tissue sample. For example, tissue imaging platform 120 may be utilized by in order to capture the image data. According to some embodiments, capturing image data may include preparing the tissue sample with a method selected from immunostaining, hybridization probes, and/or staining with tissue marking dyes. Examples of immunostaining techniques which may be utilized to prepare the tissue sample for capturing image data can include immunohistochemical staining and immunofluorescence staining. Examples of hybridization probes that may be used to prepare the tissue sample for capturing image data can include fluorescence in situ hybridization (FISH). Examples of staining with tissue-marking dyes can include using a hematoxylin and eosin (H&E) stain to prepare the tissue slide for capturing image data. Other techniques for preparing the tissue sample are not precluded.

In step 906, the method may include generating, using the captured image data, a mapping estimate of cell types for each tissue region of the tissue sample. For example, tissue imaging platform 120 may utilize machine learning model 295 to generate the mapping estimate of cell types for each tissue region of the tissue sample. The mapping estimate may be understood as the image derived prior estimate of cell type composition, or π, as it is referred to with respect to Equations (6)-(9) that are more fully described in reference to FIGS. 4A-4B. It should be noted that in some embodiments, the mapping estimate is used to parameterize the cell-type compositional estimate that is ultimately generated by the GIST methodology as described in method 900. In some embodiments the cell-type compositional estimate may be represented by a cell-type compositional matrix, and each element of the cell-type compositional matrix may be constrained by a parameterized probability distribution that specifies a prior cell type compositional estimated determined based on the mapping estimate generated in step 906. In some embodiments, the parameterized probability distribution may be beta distribution applied to each element of the cell-type compositional matrix.

In step 908, the method may include extracting a plurality of cellular analyte molecules from the tissue sample. This step may also include extracting analytes from and/or produced by cells from the tissue sample. Cellular analytes may include proteins, polypeptides, peptides, saccharides, polysaccharides, lipids, nucleic acids, and other biomolecules. Each of the plurality of cellular analyte molecules or other cellular analytes may be associated with a respective cell type of the plurality of cell types present in the tissue sample. In some embodiments, the plurality of extracted cellular analytes may be mRNA molecules, although method 900 is not limited to the extraction of mRNA molecules.

According to some embodiments, extracting the plurality of cellular analyte molecules from the tissue sample can include one or more of the following steps. For example, the method can include isolating single cells from the tissue sample using a technique such as micropipetting, cytoplasmic, laser capture microdissection, fluorescence activated cell sorting, or microfluidics. Following the isolation of single cells, the method may include lysing the single cells while preserving the plurality of cellular analyte molecules. For example, sequencer 370 of spatial transcriptomics platform 130 may be configured to extract the plurality of cellular analyte molecules (e.g., mRNA) from the tissue sample.

In step 910, the method may include generating spatially resolved transcriptomic data from the extracted plurality of cellular analyte molecules for each tissue region of the tissue sample. The spatially resolved transcriptomic data may be generated by using a spatial transcriptomics platform (e.g., spatial transcriptomics platform 130) to process the extracted plurality of cellular analyte molecules. For example, sequencer 370 of spatial transcriptomics platform 130 may be used to generate the spatially resolved transcriptomic data. According to some embodiments, the spatially resolved transcriptomic data may be represented by matrix Y as described in more detail with respect to Equation (1) and FIGS. 4A-4B.

According to some embodiments, generating the spatially resolved transcriptomic data from the extracted plurality of cellular analyte molecules can further include binding the plurality of cellular analyte molecules to a corresponding cellular analyte primer, amplifying the bound plurality of cellular analyte molecules, and preparing a sequence library of the amplified and primered cellular analyte molecules. These steps may be performed by, for example, spatial transcriptomics platform 130. In some embodiments, the spatial transcriptomic platform may be a platform selected from the commercially available Visium platform, Visium HD platform, and/or Slide-seq platform.

In step 912, the method may include determining cell-type reference data that includes gene expression by cell type for each cell type present within the tissue sample. Cell-type reference data may be determined using a latent variable model to determine the cell-type reference data directly from the spatially resolved transcriptomics data generated in step 910. In other embodiments, the cell-type reference data may be determined using a latent Dirichlet allocation model to determine the cell-type reference data directly from the spatially resolved transcriptomics data generated in step 910. In some embodiments, the cell-type reference data may be determined using non-negative matrix factorization, although any latent variable model known in the art may be used to determine cell-type reference data. In yet other embodiments, the cell-type reference data may be determined based on determining a single-cell RNA sequence dataset to estimate values of the cell-type reference data. For example, single-cell RNA-Seq platform 140 may be utilized to process the given tissue sample and generate a single-cell RNA sequence dataset. In some embodiments, the tissue sample used for generating the single-cell RNA sequence dataset may come from an adjacent tissue section, or it may come from a different patient entirely, as long as the composition of cells are similar to the tissue sample being analyzed. The resultant single-cell RNA sequence dataset may be used to infer values of the cell-type reference data. For example, the resultant cell-type reference data may be represented as W as previously described with respect to FIGS. 4A-4B, FIG. 5A, and Equations (1)-(9), and the single-cell RNA sequence dataset may be represented by Ψ. As described above with respect to Equation (3), each element of W may be estimated from Ψ using a negative binomial distribution (with overdispersion parameter ϕ_{i,k}) estimated for each gene i, in each cell type k, from the expression in each single-cell l, according to some embodiments.

In step 912, the method may include generating an output of a feature map. The feature map can be a final inferred cell type compositional map (e.g., H) for each tissue region of the tissue sample based on the spatially resolved transcriptomic data (e.g., Y), the determined cell-type reference data (e.g., W), and the mapping estimate of cell types (e.g., prior values π).

According to some embodiments, the spatially resolved transcriptomics data may be represented by a spatially resolved transcriptomics matrix (e.g., Y) that gives values of gene expression as a function of tissue region. Generating the final inferred cell type compositional map (e.g., H) may further include decomposing the spatially resolved transcriptomic matrix (e.g., Y) into a product of a cell-type signature matrix (e.g., W) and a cell-type compositional matrix (e.g., H). The values of the cell-type compositional matrix can be determined by using a Bayesian statistical model (e.g., as described by Equations (1)-(9)), whereby prior values of the final inferred cell-type compositional map are derived from the image data. According to some embodiments, the cell-type compositional matrix can be based on the mapping estimate of cell types for each tissue region of the tissue sample (e.g., prior values). According to some embodiments, the cell-type signature matrix (e.g., W) may be based on single-cell RNA sequencing data (e.g., Ψ).

Examples of the present disclosure can be implemented according to at least the following clauses:
Clause 1: A method for mapping a location of cell types within a tissue sample, the method comprising: receiving the tissue sample comprising a plurality of cell types distributed over a plurality of tissue regions of the tissue sample; capturing image data of the tissue sample; generating, using the captured image data, a mapping estimate of cell types for each tissue region of the tissue sample; extracting a plurality of cellular analyte molecules from the tissue sample, each of the plurality of cellular analyte molecules associated with a respective cell type of the plurality of cell types; generating spatially resolved transcriptomic data from the extracted plurality of cellular analyte molecules for each tissue region of the tissue sample by using a spatial transcriptomics platform to process the extracted plurality of cellular analyte molecules; determine cell-type reference data comprising gene expression by cell type for each cell type of the tissue sample; generating an output of a feature map of the tissue sample comprising a final inferred cell type compositional map for each tissue region of the tissue sample based on the spatially resolved transcriptomics data, the determined cell-type reference data, and the mapping estimate of cell types.
Clause 2: The method of clause 1, wherein capturing image data of the tissue sample comprises preparing the tissue sample with a method selected from immunostaining, including immunohistochemical staining and immunofluorescence staining, hybridization probes including Fluorescence in situ hybridization (FISH), staining with tissue-marking dyes, including a hematoxylin and eosin stain, or combinations thereof.
Clause 3: The method of clause 1, wherein generating a mapping estimate of cell types for each tissue region of the tissue sample comprises using a machine learning model selected from a convolutional neural network, a recurrent neural network, a multilayer perceptron, or combinations thereof.
Clause 4: The method of clause 1, wherein extracting the plurality of cellular analyte molecules from the tissue sample further comprises: isolating single cells from the tissue sample using a technique selected from micropipetting, cytoplasmic aspiration, laser capture microdissection, fluorescence activated cell sorting, and microfluidics; and lysing the single cells while preserving the plurality of cellular analyte molecules.
Clause 5: The method of clause 1, wherein generating spatially resolved transcriptomic data from the extracted plurality of cellular analyte molecules comprises: binding the plurality of cellular analyte molecules to a corresponding primer; amplifying the bound plurality of cellular analyte molecules; and preparing a sequence library comprising the amplified bound plurality of cellular analyte molecules.
Clause 6: The method of clause 5, wherein the spatially resolved transcriptomic data comprises a spatially resolved transcriptomics matrix comprising gene expression as a function of tissue region and generating the final inferred cell type compositional map further comprises: decomposing the spatially resolved transcriptomic matrix into a product of a cell-type signature matrix and a cell-type compositional matrix, wherein: the cell-type signature matrix comprises gene expression as a function of cell type, wherein the cell-type signature matrix is determined based on the cell-type reference data; the cell-type compositional matrix comprises cell type as a function of tissue region; and determining values of the cell-type compositional matrix by a Bayesian statistical model, whereby prior values of the final inferred cell-type compositional map are derived from the image data.
Clause 7: The method of clause 6, wherein the cell-type compositional matrix is based on the mapping estimate of cell types for each tissue region of the tissue sample.
Clause 8: The method of clause 6, wherein the cell-type signature matrix is based on single-cell RNA sequencing data.
Clause 9: The method of clause 6, wherein each element of the cell-type compositional matrix is constrained by a parameterized probability distribution that specifies a prior cell type compositional estimate determined from the mapping estimate of cell types for each tissue region of the tissue sample.
Clause 10: The method of clause 1, wherein the cell-type reference data is determined using a method selected from using a latent variable model to determine the cell-type reference data from the spatially resolved transcriptomics data or using a single-cell RNA sequence dataset to estimate values of the cell-type reference data.
Clause 11: A system for mapping a location of cell types within a tissue sample, the system comprising: one or more processors; and a non-transient memory in communication with the one or more processors and storing instructions, that when executed by the one or more processors are configured to cause the system to: receive image data of a tissue sample comprising a plurality of cell types distributed over a plurality of tissue regions of the tissue sample; generate a mapping estimate of cell types for each tissue region of the tissue sample based on the received image data; generate spatially resolved transcriptomic data for each tissue region of the tissue sample based on a plurality of cellular analyte molecules extracted from the tissue sample, wherein each of the plurality of cellular analyte molecules are associated with a respective cell type of the plurality of cell types by using a spatial transcriptomics platform to process the extracted plurality of cellular analyte molecules; determine cell-type reference data comprising gene expression by cell type for each cell type of the tissue sample; and output a feature map of the tissue sample comprising a final inferred cell type compositional map for each tissue region of the tissue sample based on the spatially resolved transcriptomic data, the determined cell-type reference data, and the mapping estimate of cell types.
Clause 12: The system of clause 11, wherein capturing image data of the tissue sample comprises preparing the tissue sample with a method selected from immunostaining, including immunohistochemical straining and immunofluorescence staining, hybridization probes including Fluorescence in situ hybridization (FISH), staining with tissue-marking dyes, including a hematoxylin and eosin stain, or combinations thereof.
Clause 13: The system of clause 11, wherein generating the mapping estimate of cell types for each tissue region of the tissue sample comprises using a machine learning model selected from a convolutional neural network, a recurrent neural network, a multilayer perceptron, or combinations thereof.
Clause 14: The system of clause 11, wherein the cell-type reference data is determined using a latent variable model to determine the cell-type reference data from the spatially resolved transcriptomics data.
Clause 15: The system of clause 11, wherein the cell-type reference data is by using a single-cell RNA sequence dataset to estimate values of the cell-type reference data.
Clause 16: The system of clause 11, wherein the spatially resolved transcriptomic data comprises a spatially resolved transcriptomics matrix comprising gene expression as a function of tissue region and generating the final inferred cell type compositional map further comprises: decomposing the spatially resolved transcriptomic matrix into a product of a cell-type signature matrix and a cell-type compositional matrix, wherein: the cell-type signature matrix comprises gene expression as a function of cell type, wherein the cell-type signature matrix is determined based on the cell-type reference data; the cell-type compositional matrix comprises cell type as a function of tissue region; and determining values of the cell-type compositional matrix by a Bayesian statistical model, whereby prior values of the final inferred cell-type compositional map are derived from the image data.
Clause 17: The system of clause 16, wherein the cell-type compositional matrix is based on the mapping estimate of cell types for each tissue region of the tissue sample.
Clause 18: The system of claim 16, wherein the cell-type signature matrix is based on single-cell RNA sequencing data.
Clause 19: The system of clause 16, wherein each element of the cell-type compositional matrix is constrained by a parameterized probability distribution that specifies a prior cell type compositional estimate determined from the mapping estimate of cell types for each tissue region of the tissue sample.
Clause 20: The system of clause 19, wherein the cell-type compositional matrix is constrained by a tuning hyperparameter which is configured to scale the respective influence of the cell-type signature matrix and the spatially resolved transcriptomic matrix on the cell-type compositional matrix.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A method for mapping a location of cell types within a tissue sample, the method comprising:
receiving the tissue sample comprising a plurality of cell types distributed over a plurality of tissue regions of the tissue sample;
capturing image data of the tissue sample;
generating, using the captured image data, a mapping estimate of cell types for each tissue region of the tissue sample;
extracting a plurality of cellular analyte molecules from the tissue sample, each of the plurality of cellular analyte molecules associated with a respective cell type of the plurality of cell types;
generating spatially resolved transcriptomic data from the extracted plurality of cellular analyte molecules for each tissue region of the tissue sample by using a spatial transcriptomics platform to process the extracted plurality of cellular analyte molecules;
determine cell-type reference data comprising gene expression by cell type for each cell type of the tissue sample; and
generating an output of a feature map of the tissue sample comprising a final inferred cell type compositional map for each tissue region of the tissue sample based on the spatially resolved transcriptomics data, the determined cell-type reference data, and the mapping estimate of cell types.

2. The method of claim 1, wherein capturing image data of the tissue sample comprises preparing the tissue sample with a method selected from immunostaining, including immunohistochemical staining and immunofluorescence staining, hybridization probes including Fluorescence in situ hybridization (FISH), staining with tissue-marking dyes, including a hematoxylin and eosin stain, or combinations thereof.

3. The method of claim 1, wherein generating a mapping estimate of cell types for each tissue region of the tissue sample comprises using a machine learning model selected from a convolutional neural network, a recurrent neural network, a multilayer perceptron, or combinations thereof.

4. The method of claim 1, wherein extracting the plurality of cellular analyte molecules from the tissue sample further comprises:
isolating single cells from the tissue sample using a technique selected from micropipetting, cytoplasmic aspiration, laser capture microdissection, fluorescence activated cell sorting, and microfluidics; and
lysing the single cells while preserving the plurality of cellular analyte molecules.

5. The method of claim 1, wherein generating spatially resolved transcriptomic data from the extracted plurality of cellular analyte molecules comprises:
binding the plurality of cellular analyte molecules to a corresponding primer;
amplifying the bound plurality of cellular analyte molecules; and
preparing a sequence library comprising the amplified bound plurality of cellular analyte molecules.

6. The method of claim 5, wherein the spatially resolved transcriptomic data comprises a spatially resolved transcriptomics matrix comprising gene expression as a function of tissue region and generating the final inferred cell type compositional map further comprises:
decomposing the spatially resolved transcriptomic matrix into a product of a cell-type signature matrix and a cell-type compositional matrix, wherein:
the cell-type signature matrix comprises gene expression as a function of cell type, wherein the cell-type signature matrix is determined based on the cell-type reference data;
the cell-type compositional matrix comprises cell type as a function of tissue region; and
determining values of the cell-type compositional matrix by a Bayesian statistical model, whereby prior values of the final inferred cell-type compositional map are derived from the image data.

7. The method of claim 6, wherein the cell-type compositional matrix is based on the mapping estimate of cell types for each tissue region of the tissue sample.

8. The method of claim 6, wherein the cell-type signature matrix is based on single-cell RNA sequencing data.

9. The method of claim 6, wherein each element of the cell-type compositional matrix is constrained by a parameterized probability distribution that specifies a prior cell type compositional estimate determined from the mapping estimate of cell types for each tissue region of the tissue sample.

10. The method of claim 1, wherein the cell-type reference data is determined using a method selected from using a latent variable model to determine the cell-type reference data from the spatially resolved transcriptomics data, or using a single-cell RNA sequence dataset to estimate values of the cell-type reference data.

11. A system for mapping a location of cell types within a tissue sample, the system comprising:
one or more processors; and
a non-transient memory in communication with the one or more processors and storing instructions, that when executed by the one or more processors are configured to cause the system to:
receive image data of the tissue sample comprising a plurality of cell types distributed over a plurality of tissue regions of the tissue sample;
generate a mapping estimate of cell types for each tissue region of the tissue sample based on the received image data;
generate spatially resolved transcriptomic data for each tissue region of the tissue sample based on a plurality of cellular analyte molecules extracted from the tissue sample, wherein each of the plurality of cellular analyte molecules are associated with a respective cell type of the plurality of cell types by using a spatial transcriptomics platform to process the extracted plurality of cellular analyte molecules;
determine cell-type reference data comprising gene expression by cell type for each cell type of the tissue sample; and
output a feature map of the tissue sample comprising a final inferred cell type compositional map for each tissue region of the tissue sample based on the spatially resolved transcriptomic data, the determined cell-type reference data, and the mapping estimate of cell types.

12. The system of claim 11, wherein capturing image data of the tissue sample comprises preparing the tissue sample with a method selected from immunostaining, including immunohistochemical straining and immunofluorescence staining, hybridization probes including Fluorescence in situ hybridization (FISH), staining with tissue-marking dyes, including a hematoxylin and eosin stain, or combinations thereof.

13. The system of claim 11, wherein generating the mapping estimate of cell types for each tissue region of the tissue sample comprises using a machine learning model selected from a convolutional neural network, a recurrent neural network, a multilayer perceptron, or combinations thereof.

14. The system of claim 11, wherein the cell-type reference data is determined using a latent variable model to determine the cell-type reference data from the spatially resolved transcriptomics data.

15. The system of claim 11, wherein the cell-type reference data is by using a single-cell RNA sequence dataset to estimate values of the cell-type reference data.

16. The system of claim 11, wherein the spatially resolved transcriptomic data comprises a spatially resolved transcriptomics matrix comprising gene expression as a function of tissue region and generating the final inferred cell type compositional map further comprises:
decomposing the spatially resolved transcriptomic matrix into a product of a cell-type signature matrix and a cell-type compositional matrix, wherein:
the cell-type signature matrix comprises gene expression as a function of cell type, wherein the cell-type signature matrix is determined based on the cell-type reference data;
the cell-type compositional matrix comprises cell type as a function of tissue region; and
determining values of the cell-type compositional matrix by a Bayesian statistical model, whereby prior values of the final inferred cell-type compositional map are derived from the image data.

17. The system of claim 16, wherein the cell-type compositional matrix is based on the mapping estimate of cell types for each tissue region of the tissue sample.

18. The system of claim 16, wherein the cell-type signature matrix is based on single-cell RNA sequencing data.

19. The system of claim 16, wherein each element of the cell-type compositional matrix is constrained by a parameterized probability distribution that specifies a prior cell type compositional estimate determined from the mapping estimate of cell types for each tissue region of the tissue sample.

20. The system of claim 19, wherein each element of the cell-type compositional matrix is constrained by a tuning hyperparameter which is configured to scale the respective influence of the cell-type signature matrix and the spatially resolved transcriptomic matrix on the cell-type compositional matrix.
